(19) United States
(12) Patent Application Publication
(10) Pub. No.: US 2004/0091887 A1
(43) Pub. Date: May 13, 2004

(54) Mucroslysin and its gene

(76) Inventors: Yaw-Wen Guo, Taipei (TW); Pei-Hsun Ho, Taoyuan City (TW)

Correspondence Address: Anna Y. Tsang, c/o BAKER & McKENZIE, 2300 Trammell Crow Center, 2001 Ross Avenue, Dallas, TX, 75201, US

(21) Appl. No.: 10/383,588

(22) Filed: Mar. 10, 2003

(30) Foreign Application Priority Data
Sep. 11, 2002 (TW) 091120741

(51) Int. Cl. C12Q 1/68; A01K 67/00; C12N 9/64; C07H 21/04

(52) U.S. Cl. 435/6; 435/69.1; 435/320.1; 435/226; 435/325; 530/388.26; 536/23.2; 800/8

(57) ABSTRACT

Novel Mucroslysin protein and nucleic acid molecules are disclosed. The invention provides purified Mucroslysin protein, fusion protein, antigenic peptides, and anti-Mucroslysin antibodies. The invention also provides isolated Mucroslysin nucleic acid molecule, recombinant vectors containing the Mucroslysin nucleic acid molecule, host cells containng the recombinant vector, and non-human transgenic animals in which the Mucroslysin nucleic acid molecule has been introduced.

### BACKGROUND OF THE INVENTION

Stroke caused by occlusive thrombi is a severe health problem for adults, especially the elderly population. Successful management of stroke depends on two important factors, timely treatment and medication. Recombinant tissue-type plasminogen activator (rt-PA) is currently the approved best choice of treatment of acute ischemic stroke and acute myocardial infarction (AMI) (Huang et al., Thromb. Res. 102, 411-425, 2001). However, rt-PA therapy is limited to those patients whose onset of stroke was within 3 hr with no formation of lytic-resistant thrombi (Huang et al., Thromb. Res. 102, 411-425, 2001). The effect of rt-PA is relatively slow and inefficient due to its indirect mode of action. rt-PA does not directly dissolve thrombi, but it activates plasmin that cleaves the thrombi by a de novo tissue plasminogen activation pathway in patients. This pathway has usually deteriorated in patients who suffer occlusive thrombus. Thus, rt-PA therapy is sometimes limited by its slow action, high incidence of asymptomatic recurrent thrombosis, poor efficacy in thrombolytic therapy in reocclusive patients, and serious hemorrhagic side effects. Other clinically available drugs, including streptokinase (SK) and urokinase (UK), have similar, or even more serious, adverse effects than rt-PA. SK and UK are associated with a relatively low recanalization rate as well as a risk of adverse effects, and most commonly, a systemic lytic state and risk of bleeding complications (Mueller et al., Med. Clin. North Am. 73, 387-407, 1989). In searching for a better drug for effective and safe thrombolytic therapy, enzymes that can directly and efficiently dissolve thrombi are under intensive investigation. Consequently, safe, non-hemorrhagic, high fibrinolytic, and fast action thrombolytic drugs are undergoing intensive studies.

Snake venoms are a rich source of the fibrinogenolytic and fibrinolytic enzymes. These enzymes are potentially important therapeutic agents for occlusive thrombi. Traditional venom isolates did demonstrate promising results in thrombolytic therapy. However, a large amount of these purified venom proteins are required due to their low potency, thus limiting their clinical application. Genetically engineered venom protein could be the solution.

Snake venoms of the Crotalidae family are known to contain a variety of proteolytic enzymes that affect blood coagulation (Jia et al., Toxicon 34, 1269-1276, 1996; Bjarnason, J. B. & Fox, J. W., Pharmacol. Ther. 62, 325-374, 1994; Gomis-Ruth et al., EMBO J. 12, 4151-4157,1993). Some enzymes, such as those in Russell's viper venom isolate (Johnson et al., Comp. Biochem. Physiol. B, 82, 647-653, 1985), accelerate blood coagulation, whereas others, such as triflavin and trigramin, have anticoagulation activities (Cercek et al., Thromb. Res. 47, 417-426, 1987). Among the enzymes that show anticoagulation activity, fibrinogenolytic and fibrinolytic enzymes are the primary subjects in many studies due to their potential as thrombolytic agents (Trikha, M., Schmitmeier, S. & Markland, F. S., Toxicon 32, 1521-1531, 1994). Many enzymes with fibrinogenolytic and fibrinolytic activities were isolated and found to be zinc-containing metalloproteinase (Hite et al., Arch. Biochem. Biophys. 308, 182-191, 1994). In addition to the potential to be a thrombolytic reagent, they are also known for their hemorrhagic activity. This makes them undesirable for clinical application. Only a few non-hemorrhagic metalloproteinases have been isolated from snake venom, such as fibrolase (Sanchez et al., Thromb. Res. 87, 289-302, 1997), H2-proteinase (Takeya et al., J. Biochem. (Tokyo) 106, 151-157, 1989), lebetase (Trummal et al., Biochim. Biophys. Acta 1476, 331-336, 2000), and vipera lebetina fibrinogenase (VIF) (Gasmi et al., Thromb. Res. 86, 233-242, 1997). These proteins are unlikely to be used in the clinic due to the difficulties in separating them from the hemorrhagic isoenzymes in snake venom.

Based on their multiple domain features, snake venom metalloproteinases can be categorized into 4 groups. The P-I group contains only a basic metalloproteinase domain, the P-II group has a disintegrin domain attached to a basic metalloproteinase domain, the P-III group has both a disintegrin and cysteine rich polypeptide domain attached to a basic metalloproteinase domain, and the P-IV group has an additional lectin-like sequence attached to the C-terminal end of the cysteine rich polypeptide of the P-III enzymes (Jia et al., Toxicon 34, 1269-1276, 1996). The hemorrhagic activity of these enzymes has been correlated with the consensus N-glycosylation sites in metalloproteinases (Hite et al., Arch. Biochem. Biophys. 308, 182-191, 1994). It has also been postulated that the large hemorrhagic metalloproteinases, i.e. those having disintegrin-like and high-cysteine domains, are more active in inducing hemorrhage than those enzymes in which comprise only the metalloproteinase domain. Nevertheless, there are metalloproteinases in snake venoms that are devoid of hemorrhagic activity (Markland, F. S., Toxicon 36, 1749-1800, 1998). The structural basis of this observation is not clear. Therefore, there is tremendous interest in the non-hemorrhagic action of metalloproteinase and their potential for therapeutic application.

Snake venoms with non-hemorrhagic thrombolytic activities might avoid the disadvantages of rt-PA, UK, and SK. These non-hemorrhagic fibrinogenases include fibrolase (Sanchez et al., Thromb. Res. 87, 289-302, 1997), H2-proteinase (Takeya et al., J. Biochem. (Tokyo) 106, 151-157, 1989), lebetase (Trummal et al., Biochim. Biophys. Acta 1476, 331-336, 2000), vipera lebetina fibrinogenase (VIF) (Gasmi et al., Thromb. Res. 86, 233-242, 1997), and atroase (Willis et al., Thromb. Res. 53, 19-29, 1989). These proteins are unlikely to be used in the clinic due to the fact that it is very difficult to isolate a large quantity of highly purified venom protein with traditional biochemical methodology. For instance, to obtain a large amount of snake venom and to separate the desired proteins from their hemorrhagic isoenzymes is not an easy task. The other drawback of using the venom isolate proteins is that they are at least 5 to 6 times less potent than the recombinant Mucroslysin protein (Sanchez et al., Thromb. Res. 87, 289-302, 1997; Willis et al., Thromb. Res. 53, 19-29, 1989). It is conceivable that the use of genetically engineered proteins might overcome these difficulties. Some genetically engineered venom proteins, such as MT-C, MT-d-I, and MT-d-II, did show collagenolytic and gelatinolytic activity in vitro (Jeon, 0. H. & Kim, D. S., Biochem. Mol. Biol. Int. 47, 417-425, 1999; Jeon, 0. H, & Kim, D. S., Eur J. Biochem. 263, 526-533, 1999). However, there is no report on genetically engineered venom proteins regarding this thrombolytic activity. The obstacle of this approach lies in reconstitution of the biological activity. There are twenty-two cysteinyl residues left in the sequence of Mucroslysin which could form a maximum number of 11 intermolecular disulfide bonds. These disulfide bonds make it difficult to refold the Mucroslysin protein successfully. The venom proteins that are successfully refolded contain a maximum of 7 disulfide bonds (Chang et al., Biochem. Biophys. Res. Commun. 225, 990-996, 1996).

### SUMMARY OF THE INVENTION

The present invention relates to the discovery of a gene, named Mucroslysin, of a non-hemorrhagic thrombolytic snake venom protein from Trimeresurus mucrosquamatus. Mucroslysin gene is isolated, sequenced, characterized, cloned, and expressed, and the biological function of its protein is reconstituted and analyzed. This genetically engineered snake venom protein, designated as Mucroslysin, demonstrated a powerful thrombolytic effect within 15 min without hemorrhagic side effects. In addition to the potent thrombolytic effect, Mucroslysin exhibits a unique feature that makes it an attractive candidate for treating occlusive thrombi: it directly lyses the thrombi compared to the indirect action of the clinically available drugs. Therefore, Mucroslysin has the potential to become a very useful alternative to the current treatment of stroke.

The present invention is based, at least in part, on the discovery of the gene encoding Mucroslysin (SEQ ID NO:1) which has 1,389 nucleotides and encodes 463 amino acids (SEQ ID NO:2). Mucroslysin contains a metalloproteinase domain (SEQ ID NO:3, SEQ ID NO:4) and a disintegrin domain (SEQ ID NO:5, SEQ ID NO:6).

A longer form of Mucroslysin, including the untranslated region of the Mucroslysin gene at its 5′ and 3′ end is depicted in SEQ ID NO:7. The resulting protein contains a signal peptide region and has a total of 481 amino acids (SEQ ID NO:8).

The invention features a nucleic acid molecule, which is at least 50% (or 60%, 70%, 75%, 80%, 85%, 90%, 95%, 96%, 97%, or 98%) identical to the nucleotide sequence shown in SEQ ID NO:1, SEQ ID NO:3, SEQ ID NO:5, or a complement thereof.

The invention features a nucleic acid molecule, which includes a nucleotide sequence encoding a protein having an amino acid sequence that is at least 98% or 99% identical to the amino acid sequence of SEQ ID NO:2 or SEQ ID NO:4.

The invention also features a Mucroslysin nucleic acid molecule with the nucleotide sequence shown in SEQ ID NO:1, SEQ ID NO:3, or SEQ ID NO:5.

The invention includes a nucleic acid molecule, which encodes an allelic variant, naturally occurring or artificial, of a polypeptide comprising the amino acid sequence of SEQ ID NO:2, wherein the nucleic acid molecule hybridizes to nucleic acid molecule comprising SEQ ID NO:1 under stringent conditions. The invention further includes a nucleic acid molecule, which encodes an allelic variant, naturally occurring or artificial, of a polypeptide comprising the amino acid sequence of SEQ ID NO:4, wherein the nucleic acid molecule hybridizes to nucleic acid molecule comprising SEQ ID NO:3 under stringent conditions. The invention also encompasses a nucleic acid molecule, which encodes an allelic variant, naturally occurring or artificial, of a polypeptide comprising the amino acid sequence of SEQ ID NO:6, wherein the nucleic acid molecule hybridizes to nucleic acid molecule comprising SEQ ID NO:5 under stringent conditions.

Also within the invention are: a purified Mucroslysin protein having an amino acid sequence that is at least about 97%, 98%, or 99% identical to the amino acid sequence of SEQ ID NO:2; a purified polypeptide having an amino acid sequence that is at least about 98%, 99%, 99.5% or 99.9% identical to SEQ ID NO:4.

Also within the invention is an allelic variant, naturally occurring or artificial, of a polypeptide that includes the amino acid sequence of SEQ ID NO:2, wherein the polypeptide is encoded by a nucleic acid molecule which hybridizes to a nucleic acid molecule comprising SEQ ID NO: 1 under stringent conditions. The invention also includes an allelic variant, naturally occurring or artificial, of a polypeptide that includes the amino acid sequence of SEQ ID NO:4, wherein the polypeptide is encoded by a nucleic acid molecule which hybridizes to a nucleic acid molecule comprising SEQ ID NO: 3 under stringent conditions. The invention further includes an allelic variant, naturally occurring or artificial, of a polypeptide that includes the amino acid sequence of SEQ ID NO:6, wherein the polypeptide is encoded by a nucleic acid molecule which hybridizes to a nucleic acid molecule comprising SEQ ID NO: 5 under stringent conditions.

The invention includes purified polypeptides, which have the amino acid sequences of SEQ ID NO:2, SEQ ID NO:4, SEQ ID NO:6, or SEQ ID NO:8 respectively, or sequences with at least one conservative amino acid substitutions.

Another aspect of the invention provides a vector, e.g. a recombinant expression vector, comprising nucleic acid molecule of the invention (SEQ ID NO:1, SEQ ID NO:3, SEQ ID NO:5.). Preferred protein and polypeptides possess at least one biological activity possessed by naturally occurring Mucroslysin proteins, e.g., (1) cleave fibrinogen protein and binding fibrinogen.

The invention further features antibodies, monoclonal or polyclonal, that specifically bind proteins encoded by SEQ ID NO:2, SEQ ID NO:4, SEQ ID NO:6, and SEQ ID NO:8. In addition, these proteins can be incorporated into pharmaceutical compositions, which optionally include pharmaceutically acceptable carriers.

In another aspect, the invention provides method of purifying protein encoded by SEQ ID NO:2, SEQ ID NO:4, SEQ ID NO:6 or SEQ ID NO:8 from a biological sample containing such proteins by providing an affinity matrix comprising the antibodies specific for these proteins and contact the biological sample with the affinity matrix in order to select out the desired protein.

### BRIEF DESCRIPTION OF THE DRAWINGS

FIG. 1 depicts the cDNA sequence (SEQ ID NO:1) of Mucroslysin.

FIG. 2 depicts the predicted amino acid sequence (SEQ ID NO:2) of Mucroslysin.

FIG. 3 depicts the cDNA sequence of the metalloproteinase domain (SEQ ID NO:3) of Mucroslysin.

FIG. 4 depicts the predicted amino acid sequence of the metalloproteinase domain (SEQ ID NO:4) of Mucroslysin.

FIG. 5 depicts the cDNA sequence of the disintegrin domain (SEQ ID NO:5) of Mucroslysin.

FIG. 6 depicts the predicted amino acid sequence of the disintegrin domain (SEQ ID NO:6) of Mucroslysin.

FIG. 7 depicts the cDNA sequence of SEQ ID NO:7 and the amino acid sequence of SEQ ID NO:8. Dash line under the amino acid sequence indicates the putative signal peptide. The Kozak sequence aaaATGA and the polyadenylation signal, AATAAA, are boxed. The conserved sequences at the zymogen region, zinc ion binding region, and the RGD consensus region are underlined. The arrows are the GSP5b and GST3b primers.

FIG. 8 depicts the multiple alignment and sequence comparison of SEQ ID NO:8 and other fibrinogenase enzymes. The solid box shows the residues conserved in zinc ion consensus binding region and the RGD-disintegrin consensus domain. The dotted-line box represents a consensus domain of the zymogen region. The 23 cysteine residues of the protein are highlighted by asterisks.

FIG. 9 depicts the result of tissue-specific transcriptional analysis of SEQ ID NO:7. Northern blots were carried out with [32 P] labeled SEQ ID NO:7 cDNA probe. The sizes of the RNA markers and the organ from which the RNAs are extracted are indicated. The same blot was probed with a [³²P] labeled 18S ribosomal RNA.

FIG. 10 depicts the result of tricine SDS-PAGE and immunological analysis of the recombinant protein generated from Mucroslysin cDNA. Section (A) shows the protein profiles of expressed protein analyzed on 10% tricine SDS-PAGE with coomassie blue and section (B) shows Western blotting analysis of the recombinant protein purified from His25 bind resin. Lane 1 is molecular weight marker. Lane 2 is pET21a(I), a vector control induced with IPTG. Lane 3 is pSEQ ID NO:7. Lane 4 is pSEQ ID NO:7(I), a his-bind resin purified recombinant protein without/with IPTG induction for 2 hrs.

FIG. 11 depicts the result of time-course study of fibrinogenolytic activity of the refolded Mucroslysin protein.

FIG. 12 depicts the result of the in vivo thrombolytic assay of Mucroslysin protein on artificial thrombus. Film (A) is the angiogram taken after thrombus induction. The artificial thrombus completely occludes anterior blood flow at time zero. Film (B) is the angiogram taken at 15 minutes after injection of 1.0 mg/kg body weight of Mucroslysin protein. The arrows indicate the region of recanalization.

FIG. 13 depicts the result of Tricine SDS-PAGE and immunological analysis of the recombinant fibrinlysin protein. (A) is the protein profiles of expressed fibrinlysin analyzed on 10% tricine SDS-PAGE with Coomassie blue and (B) is the Western blotting analysis of the recombinant fibrinlysin purified from His-bind resin. The arrow indicates the expressed fibrinlysin protein. Lane 1, protein molecular weight marker (25.4-61.5 kDa). Lane 2, pET32a(I) is vector control induced with IPTG; Lane 3, pFibrinlysin and Lane 4, pFibrinlysin (I) are His-bind resin purified recombinant fibrinlysin proteins with/without IPTG induction for 2 hrs, respectively.

FIG. 14 depicts the result of the time-course study of fibrinogenolytic activity of the refolded fibrinlysin protein.

FIG. 15 depicts the result of hemorrhagic activity analysis of the recombinant fibrinlysin protein. The black arrow indicated the injection site of a high dose recombinant protein showing an induced hemorrhagic spot less than 3 mm³. The white arrow indicated the positive control of injecting 1 to 20 diluted T. mucrosquamatus venom protein.

### DETAILED DESCRIPTION OF THE INVENTION

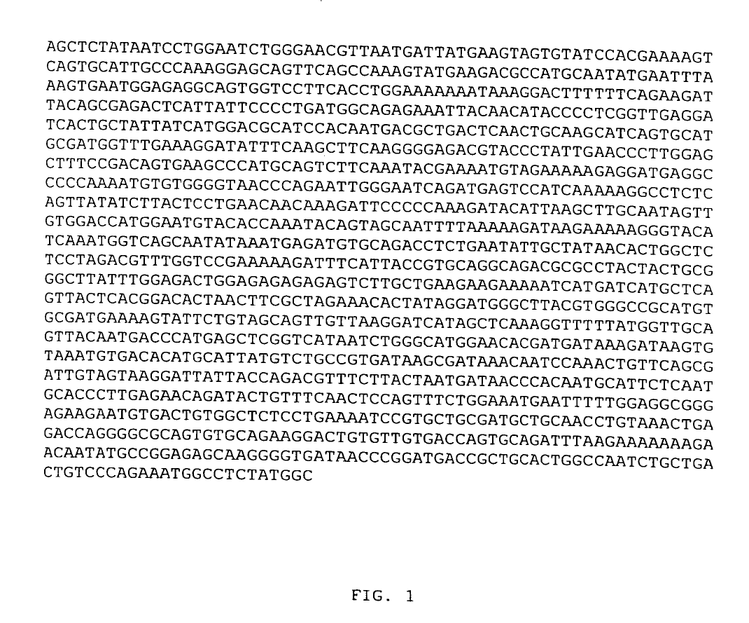

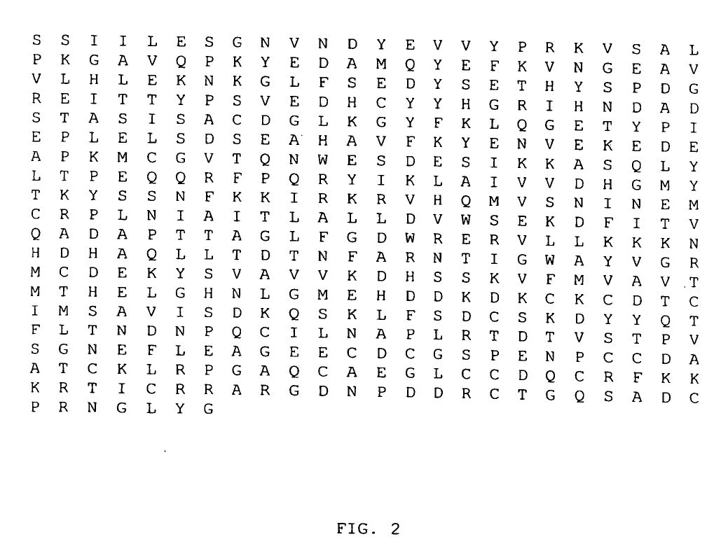

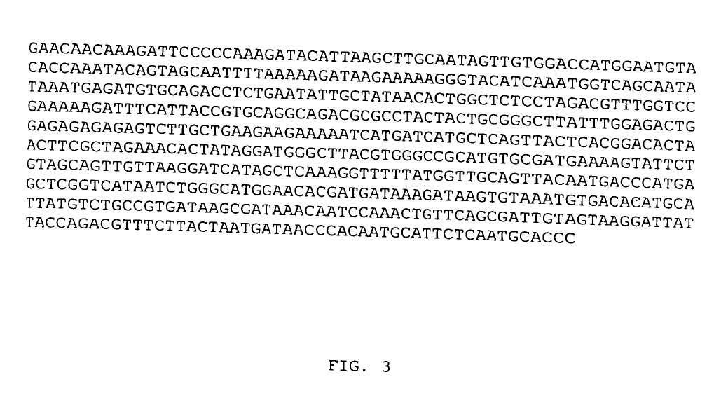

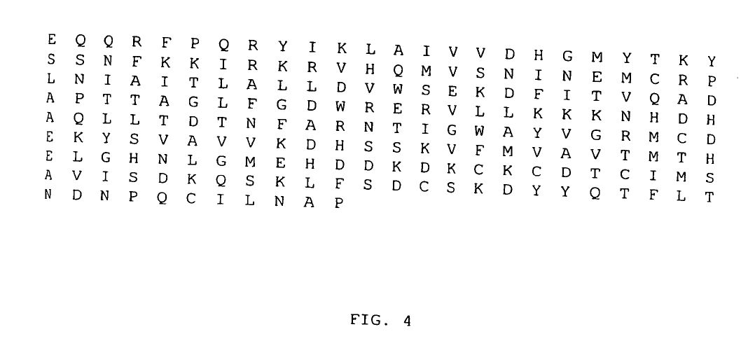

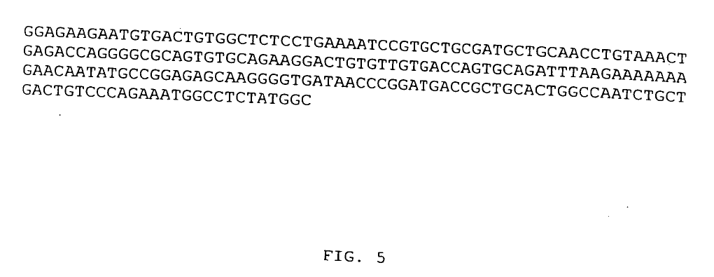

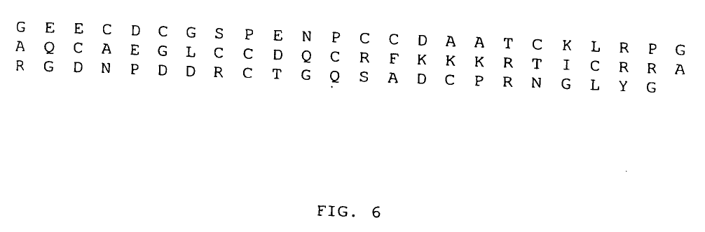

The present invention relates to the discovery of a cDNA molecule region, encoding a snake venom protein, that exhibits a potent thrombolytic effect, said protein region is designated as Mucroslysin. A nucleotide sequence encoding Mucroslysin is shown in FIG. 1 (SEQ ID NO:1). A predicted amino acid sequence of the Mucroslysin protein is also shown in FIG. 2 (SEQ ID NO:2). The Mucroslysin protein has a metalloproteinase domain which nucleotide sequence and amino acid sequence are shown in FIG. 3 (SEQ ID NO:3) and FIG. 4 (SEQ ID NO:4) respectively. The protein encoded by SEQ ID NO: 4 is also herein referred to as “fibrinlysin” in the alternative. The nucleotide sequence and amino acid sequence of the disintegrin domain of Mucroslysin are shown in FIG. 5 (SEQ ID NO:5) and FIG. 6 (SEQ ID NO: 6) respectively.

The Mucroslysin cDNA of FIG. 1 (SEQ ID NO:1), which is approximately 1389 nucleotides long, encodes a protein having 463 amino acids. The metalloproteinase is approximately 609 nucleotides long, and the disintegrin domain has approximately 213 nucleotides.

The Mucroslysin protein is a member of a family of molecules (the snake venom protein family) having certain conserved structural and functional features. The term “family” when referring to the protein and nucleic acid molecules of the invention is intended to mean two or more proteins or nucleic acid molecules having a common structural domain and having sufficient amino acid or nucleotide sequence identity as defined herein. Such family members can be naturally occurring and can be from either the same or different species. For example, a family can contain a protein of snake origin and a homologue of that protein of murine origin etc.

The term “sufficiently identical” as used herein refers to a first amino acid or nucleotide sequence which contains a sufficient or minimum number of identical or equivalent (e.g. an amino acid residue which has a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having about 85%, 95% or 99% identity are defined herein as sufficiently identical.

1. Isolated Nucleic Acid Molecules

One aspect of the invention pertains to isolated nucleic acid molecules that encode the Mucroslysin protein or biologically active portions thereof, as well as nucleic acid molecules sufficient for use as hybridization probes to identify Mucroslysin encoding nucleic acids (e.g. Mucroslysin mRNA) and fragments for use as PCR primers for the amplification or mutation of Mucroslysin nucleic acid molecules. As used herein, the term “nucleic acid molecule” is intended to include DNA molecules (e.g. cDNA or genomic DNA) and RNA molecules (e.g. mRNA) and analogs of the DNA or RNA generated using nucleotide analogs. The nucleic acid molecule can be single-stranded or double-stranded, but preferably is double-stranded DNA.

An “isolated” nucleic acid molecule is one, which is separated from other nucleic acid molecules which are present in the natural source of the nucleic acid and which encode all necessary sequences for transcription and translation of protein. Preferably, an “isolated” nucleic acid is free of sequences, preferably protein encoding sequences, which naturally flank the nucleic acid, i.e., sequences located at the 5′ and 3′ ends of the nucleic acid, in the genomic DNA of the organism from which the nucleic acid is derived, and, in addition, preferably, the isolated nucleic acids does not span beyond the amino acid encoding region of a single gene. Moreover, an “isolated” nucleic acid molecule, such as a cDNA molecule, can be substantially free of other cellular material, or culture medium when produced by recombinant techniques, or substantially free of chemical precursors or other chemical when chemically synthesized.

A nucleic acid molecule of the present invention, e.g., nucleic acid molecule having the nucleotide sequence of SEQ ID NO: 1, SEQ ID NO:3, SEQ ID NO:5, or a complement of any of these nucleotide sequences, can be isolated using standard molecular biological techniques and the sequence information provided herein. Using all or portion of the nucleic acid sequence of SEQ ID NO: 1, SEQ ID NO:3, or SEQ ID NO:5, as a hybridization probe, Mucroslysin nucleic acid molecules can be isolated using standard hybridization and cloning techniques (e.g. as described in Sambrook et al., eds., Molecular Cloning: A Laboratory Manual 2^{nd}, eds., Cold Spring harbor Laboratory, Cold Spring Harbor Laboratory Press, Cold Spring Harbor, N.Y., 1989).

A nucleic acid of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to Mucroslysin nucleotide sequences can be prepared by standard synthetic techniques, e.g., using an automated DNA synthesizer.

The invention also encompasses nucleic acid molecules that differ from the nucleotide sequence of SEQ ID NO:1, SEQ ID NO:3, or SEQ ID NO:5, due to the degeneracy of the genetic code and thus encode the same Mucroslysin protein as that which is encoded by the nucleotide sequence shown in SEQ ID NO:1, SEQ ID NO:3, or SEQ ID NO:5.

In addition to the Mucroslysin nucleotide sequence shown in SEQ ID NO:1, SEQ ID NO:3, or SEQ ID NO:5, it will be appreciated by those skilled in the art that DNA sequence polymorphisms that lead to changes in the amino acid sequences of Mucroslysin may exist within a population, such as the snake population. Such genetic polymorphism in the Mucroslysin gene may exist among individuals within a population due to natural allelic variation. As used herein, the terms “gene” and “recombinant gene” refer to nucleic acid molecules comprising an open reading frame encoding a Mucroslysin protein, preferably a non-mammalian Mucroslysin protein. Such natural allelic variations can typically result in 0.1-5% variance in the nucleotide sequence of the Mucroslysin gene. Any and all such nucleotide variations and resulting amino acid polymorphisms in Mucroslysin that are the result of natural allelic variation and that do not alter the functional activity of Mucroslysin are intended to be within the scope of the invention.

Moreover, nucleic acid molecules encoding Mucroslysin protein from other species, i.e. Mucroslysin homologues, which have a nucleotide sequence, which differs from that of the Mucroslysin from Trimeresurus mucrosquamatus, are intended to be within the scope of the invention. Nucleic acid molecules corresponding to natural allelic variants and homologues of the Mucroslysin cDNA of the invention can be isolated based on their identity to the T. mucrosquamatus Mucroslysin nucleic acid disclosed herein using the T. mucrosquamatus cDNAs, or a portion thereof, as a hybridization probe according to standard hybridization techniques under stringent hybridization conditions.

In another preferred embodiment, an isolated nucleic acid molecule of the invention comprises a nucleic acid molecule, which is a complement of the nucleotide sequence shown in SEQ ID NO:1, SEQ ID NO:3, or SEQ ID NO:5. A nucleic acid molecule which is complementary to a given nucleotide sequence is one which is sufficiently complementary to the given nucleotide sequence that it can hybridize to the given nucleotide sequence thereby forming a stable duplex.

Moreover, the nucleic acid molecule of the invention can comprise only a portion of a nucleic acid sequence encoding Mucroslysin, for example, a fragment that can be used as a probe or primer or a fragment encoding a biologically active portion of Mucroslysin. The nucleotide sequence determined from cloning the Mucroslysin gene allows for the generation of probes and primers designed for use in identifying and/or cloning Mucroslysin homologues in other cell types, such as from other tissues, as well as Mucroslysin homologues in other species. The probe/primer typically comprises substantially purified oligonucleotide. The oligonucleotide typically comprises a region of the nucleotide sequence that hybridizes under stringent conditions to at least about 100, preferably about 200, more preferably about 300, 400, 500, 600, 700, 800, 850, or 870 consecutive nucleotides of the sense or antisense sequence of SEQ ID NO:1, SEQ ID NO:3, or SEQ ID NO:5, or of a naturally occurring mutant of SEQ ID NO:1, SEQ ID NO:3, or SEQ ID NO:5.

Probes based on the Mucroslysin nucleotide sequence can be used to detect transcripts or genomic sequences encoding the same or identical proteins. The probe comprises a label group attached thereto, e.g., a radioisotope, a fluorescent compound, an enzyme, or an enzyme co-factor. Such probes can be used as a part of an assaying kit for identifying cells or tissue, which expresses Mucroslysin or biologically active portion thereof.

A nucleic acid fragment encoding a “biologically active portion” of Mucroslysin can be prepared by isolating a portion of SEQ ID NO:1, SEQ ID NO:3, or SEQ ID NO:5, which encodes a polypeptide having the respective biological activity, expressing the encoded portion of the protein (e.g. by recombinant expression in vitro) and assessing the activity of the encoded portion of the protein. For example, a nucleic acid fragment encoding a biologically active portion of Mucroslysin includes the metalloproteinase and disintegrin domains, i.e. SEQ ID NO:3 and SEQ ID NO:5.

In another embodiment of the invention, an isolated nucleic acid molecule of the invention is at least 100, 150, 200, 300, 400, 500, 600, 700, 800, 850, or 870 nucleotides in length and hybridize under stringent conditions to the nucleic acid molecule comprising the nucleotide sequence, preferably the coding sequence of SEQ ID NO:1, SEQ ID NO:3, or SEQ ID NO:5.

As used herein, the term “hybridizes under stringent conditions” is intended to describe conditions for hybridization and washing under which nucleotide sequences at least 60% (65%, 70%, 75%, 80%, 85%, 90%, 95%, 98% preferably 99%) identical to each other typically remain hybridized to each other. Such stringent conditions are known to those skilled in the art and can be found in Current Protocols in Molecular Biology, John Wiley & Sons, N.Y. 6.3.1-6.3.6, 1989. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of the Mucroslysin sequence that may exist in the population, the skilled artisan will further appreciate that changes can be introduced by mutation into the nucleotide sequence of SEQ ID NO:1, SEQ ID NO:3, and SEQ ID NO:5, thereby leading to changes in the amino acid sequence of the encoded Mucroslysin protein, without altering the functional ability of the Mucroslysin protein. For example, one can make nucleotide substitutions leading to amino acid substitution at “non-essential” amino acid residues. A “non-essential” amino acid residue is a residue that can be altered from the wild-type sequence of Mucroslysin (SEQ ID NO:1, SEQ ID NO:3, and SEQ ID NO:5) without altering the biological activity, whereas an “essential” amino acid residue is required for biological activity. Thus, amino acid residues that are conserved among the Mucroslysin proteins of various species are predicted to be particularly unamenable to alteration.

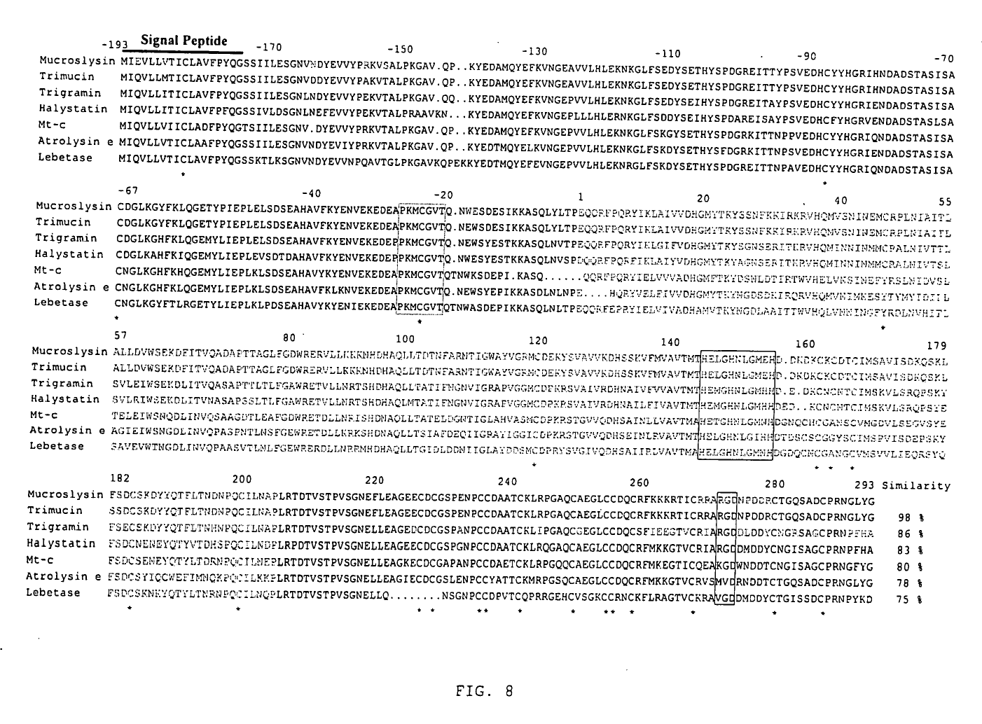

For example, preferred Mucroslysin protein of the present invention contain a Mucroslysin contains a signal peptide consisting of 18 conserved amino acids. Following the signal peptide is the zymogen sequence consisting of 171 conserved amino acids. The highly conserved sequence PKMCGVT is located near the end of the zymogen region. The proteinase domain contains 203 amino acid residues including seven cysteine residues, six of which are proposed to be involved in intrachain disulfide bonds (Zhu et al., Acta Crystallogr. D Biol. Crystallogr 55, 1834-1841, 1999). Like other snake venom metalloproteinase, the active-site consensus motif HEXXHXXGXXH is found in the proteinase domain. However, there are no N-glycosylation sites in the metalloproteinase region of Mucroslysin. The lack of N-glycosylation may be associated with the non-hemorrhagic property of venom metalloproteinases (Nikai et al., Arch. Biochem. Biophys. 378, 6-15, 2000). Following the proteinase domain is a 16-amino acids region that joins the second domain, the disintegrin domain of the Mucroslysin. The refolded Mucroslysin protein possesses anti-platelet activity, implying (data not shown) that the disintegrin domain of Mucroslysin might serve as an inhibitor of integrins to block platelet adhesion to fibrinogen. Unlike most of the hemorrhagic P-II metalloproteinases, Mucroslysin has a typical RGD cell-binding consensus sequence instead of an RGD-like sequence in the disintegrin domain (See FIG. 8). The typical RGD consensus sequence in the disintegrin domain could play a role in reducing the complication of rethrombosis or bringing the Mucroslysin protein to the thrombus and enhancing the thrombolytic activity on site by the RGD-platelet interaction (Huang et al., Thromb. Res. 102, 411-425, 2001; Takeya et al., J. Biochem. (Tokyo) 106, 151-157, 1989). The conserved domains are less likely to be amenable to mutation. Other amino acid residues, however, (e.g., those that are not conserved or only semi-conserved among Mucroslysin of various species) may not be essential for activity and thus are likely to be amenable to alteration.

Accordingly, another aspect of the invention pertains to nucleic acid molecules encoding the Mucroslysin protein that contain changes in amino acid residues that are not essential for activity. Such Mucroslysin protein differs in amino acid sequence from SEQ ID NO:2, SEQ ID NO:4, and SEQ ID NO:6 and yet retain biological activity. In one embodiment, the isolated nucleic acid molecule includes a nucleotide sequence encoding a protein that includes an amino acid sequence that is at least about 90%, 98%, or 99% identical to the amino acid sequence of SEQ ID NO:2, SEQ ID NO:4, and SEQ ID NO:6.

An isolated nucleic acid molecule encoding a Mucroslysin protein having a sequence which differs from that of SEQ ID NO:2, SEQ ID NO:4, or SEQ ID NO:6 respectively can be created by introducing one or more nucleotide substitutions, additions, or deletions into the nucleotide sequence of SEQ ID NO:1, SEQ ID NO:3, and SEQ ID NO:5 respectively such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitutions” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Therefore, a predicted nonessential amino acid residue in Mucroslysin protein is preferably replaced with another amino acid residue from the same side chain family. Alternatively, mutations can be introduced randomly along all or part of a Mucroslysin coding sequence such as by saturation mutagenesis, and the resultant mutant can be screened for Mucroslysin biological activity to identify mutants that retain activity. Following mutagenesis, the encoded protein, can be expressed recombinantly and the activity of the protein can be determined.

In a preferred embodiment, that mutant Mucroslysin protein can be assayed for the ability to form proteins and protein interactions with fibrinogen.

The present invention relates to antisense nucleic acid molecules, i.e., molecules that are complementary to sense nucleic acid encoding a protein, for example, complementary to the coding strand of a double-stranded cDNA molecule or complementary to an mRNA sequence. Accordingly, antisense nucleic acids can form hydrogen bond to a sense nucleic acid. The antisense nucleic acid can be complementary to the entire Mucroslysin coding strand, or to only portion thereof, e.g., all or part of the protein coding region or open reading frame.

Given the coding strand sequences encoding Mucroslysin disclosed herein, i.e., SEQ ID NO: 1, SEQ ID NO: 3, and SEQ ID NO: 5, antisense nucleic acid of the invention can be designed based on the base pairing rules of Watson and Crick. The antisense nucleic acid molecule can be complementary to the entire coding region of Mucroslysin mRNA, but more preferably is an oligonucleotide, which is antisense to only a portion of the coding region of a Mucroslysin mRNA. For example, the antisense oligonucleotide can be complementary to the region within the active site of the metalloprotease domain and the binding motif of the disintegrin domain. An antisense oligonucleotide can be, for example, about 5, 10, 20, 25, 30, 35, 40, 45, or 50 nucleotides in length. An antisense nucleic acid of the present invention can be constructed using chemical synthesis and enzymatic ligation reactions using procedures known in the art. For example, an antisense nucleic acid can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carbosymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methyl guanine, 3-methylcyctosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosne, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation, i.e., RNA transcribed from the inserted nucleic acid will be of an antisense orientation to a target nucleic acid of interest.

The antisense nucleic acid molecules of the invention are typically administered to a subject or generated in situ such that they hybridize with or bind to cellular mRNA and/or genomic DNA encoding a Mucroslysin protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies, which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15, 6625-6641, 1987). The antisense nucleic acid molecule can also comprise a 2′-o-methylribonucleotide (Inoue et al., Nucleic Acids Res. 15, 6131-6148, 1987) or a chimeric RNA-DNA analogue (Inoue et al., FEBS Lett. 215, 327330, 1987).

In preferred embodiments, the nucleic acid molecules of the invention can be modified at the base moiety, sugar moiety or phosphate backbone to improve, e.g., the stability in hybridization, or solubility of the molecule. For example, the deoxyribose phosphate backbone of the nucleic acids can be modified to generate peptide nucleic acids (see Hyrup et al., Bioorganic & Medicinal Chemistry 4(1), 5-23, 1996). As used herein, the terms “peptide nucleic acids” or “PNAs” refer to nucleic acid mimics, e.g., DNA mimics, in which the deoxyribose phosphate backbone is replaced by pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al., supra, 1996; Perry-Okeefe et al., Proc. Nail. Acad. Sci. USA 93, 14670-675, 1996.

PNAs of Mucroslysin can be used in therapeutic and diagnostic applications. For example, PNAs can be used as antisense or antigen agents for sequence specific modulation of gene expression by e.g., inducing transcription or translation arrest or inhibiting replication. PNAs of Mucroslysin can also be used, e.g., in the analysis of single base pair mutations in a gene by, e.g., PNA directed PCR clamping; as artificial restriction enzymes when used in combination with other enzymes, e.g., SI nucleases (Hyrup, supra, 1996; or as probes or primers for DNA sequence and hybridization (Hyrup et al., supra, 1996; Perry-Okeefe et al., Proc. Natl. Acad. Sci. USA 93, 14670-675, 1996).

In another embodiment, PNAs of Mucroslysin can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of Mucroslysin can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al., supra, 1996). The synthesis of PNA-DNA chimeras can be performed as described in Hyrup et al., supra, 1996 and Fin et al., Nucleic Acids Research 24(17), 3357-63, 1996. For example, a DNA chain can by synthesized on a solid support using standard phosphoramidite coupling chemistry and modified nucleoside analogs, e.g., 5′-4-(methoxytrityl)amino-5′-deoxy-thymidine phosophoramidite, can be used as a between the PNA and the 5′ end of DNA (Mag et al., Nucleic Acid Res. 17, 5973-88, 1989). PNA monomers are then coupled in a stepwise manner to produce a chimeric molecule with a 5′ PNA segment and a 3′ DNA segment (Fin et al., Nucleic Acids Research 24(17), 3357-63, 1996). Alternatively, chimeric molecules can by synthesized with a 5′ DNA segment and a 3′ PNA segment (Peterser et al., Bioorganic Med. Chem. Lett. 5, 1119-1124,1975).

In other embodiments, the oligonucleotide may include other appended groups such as peptides (e.g., for targeting host cell receptors in vivo), or agents facilitating transport across the cell membrane (see, e.g., Letsinger et al., Proc. Natl. Acad. Sci. USA 86, 6553-6556, 1989; Lemaitre et al., Proc. Natl. Acad. Sci. USA 84, 648-652, 1987; PCT publication No. WO 88/09810) or the blood-brain barrier (see, e.g., PCT Publication NO. WO 89/10134). In addition, oligonucleotides can be modified with hybridization-triggered cleavage agents (see, e.g., Krol et al., Bio/Techniques 6, 958-976, 1988) or intercalating agents (see, e.g., Zon, Pharm. Res. 5, 539-549, 1988). To this end, the oligonucleotide may be conjugated to another molecule, e.g., a peptide, hybridization triggered cross-linking agent, transport agent, hybridization-triggered cleavage agent etc.

II. Isolated Mucroslysin or SEQ ID NO:8 Proteins and Anti-Mucroslysin or SEQ ID NO:8 Antibodies

The present invention also relates to purified Mucroslysin or SEQ ID NO:8 proteins and biologically active portions thereof, as well as polypeptide fragments suitable for use as immunogens to raise anti-Mucroslysin or SEQ ID NO:8 antibodies. In one embodiment, native Mucroslysin or SEQ ID NO:8 proteins can be isolated from cells, tissues, or body fluid sources by an appropriate purification scheme using standard protein purification techniques. In another embodiment, Mucroslysin or SEQ ID NO:8 proteins are produced by recombinant DNA techniques. Alternative to recombinant expression, a Mucroslysin or SEQ ID NO:8 protein or polypeptide can be synthesized chemically using standard peptide synthesis techniques.

An “isolated” or “purified” protein or biologically active portion thereof is substantially free of cellular material on other contaminating proteins from the cell, tissue, or body fluid sources from which a Mucroslysin or SEQ ID NO:8 protein is derived or substantially free from chemical precursors or other chemicals when chemically synthesized. The term “substantially free of cellular material” includes preparations of Mucroslysin or SEQ ID NO:8 protein in which the protein is separated from cellular components of the cell from which it is isolated or recombinantly produced. Thus, Mucroslysin or SEQ ID NO:8 protein that is substantially free of cellular material includes preparations of Mucroslysin or SEQ ID NO:8 protein having less than about 30%, 20%, 10%, or 5% (by dry weight) of non-Mucroslysin or SEQ ID NO:8 protein (also referred to herein as a “contaminating protein”). When a Mucroslysin or SEQ ID NO:8 protein or biologically active portion thereof is recombinantly produced, it is also preferably substantially free of chemical precursors or other chemicals, i.e., it is separated from chemical precursors or other chemicals which are involved in the synthesis of the protein. Accordingly, such preparations of Mucroslysin or SEQ ID NO:8 protein have less than about 30%, 20%, 10%, or 5% (by dry weight) of chemical precursors or non-Mucroslysin or SEQ ID NO:8 chemicals.

Biologically active portions of a Mucroslysin or SEQ ID NO:8 protein include peptides comprising amino acid sequence sufficiently identical to or derived from the amino acid sequence of a Mucroslysin or SEQ ID NO:8 protein or portions thereof disclosed in SEQ ID NO: 2, SEQ ID NO:4, SEQ ID NO:6, or SEQ ID NO:8 which exhibits at least one activity of a Mucroslysin or SEQ ID NO:8 protein. A biologically active portion of a Mucroslysin or SEQ ID NO:8 protein can be a polypeptide which is, for example, 10, 25, 50, 100 or more amino acids in length. Other biologically active portions can be prepared by recombinant techniques and evaluated for one or more of the functional activities of a native Mucroslysin or SEQ ID NO:8 protein.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent homology between two sequences can be accomplished using a mathematical algorithm. A preferred, non-limiting example of a mathematical algorithm utilized for the comparison of two sequences is the algorithm of Karlin and Altschul, Proc. Nat'l Acad. Sci. USA 87, 2264-2268, 1990, modified as in Karlin and Atlschul, J. Mol. Biol. 215, 403-410, 1990. BLAST nucleotide searches can be performed with the NBLAST program, score=100, word length=12 to obtain nucleotide sequences homologous to Mucroslysin nucleic acid molecule of the invention. BLAST protein searches can be performed with the XBLAST program, score=50, word length=3 to obtain nucleotide sequences analogous to Mucroslysin protein molecule of the invention. To obtain gapped alignments for comparison purposes, Gapped Blast can be utilized as described in Altschul et al., Nucleic Acids Res. 25, 3389-3402, 1997. When utilized BLAST and Gapped BLAST programs, the default parameters of the respective programs can be used.

The invention also provides Mucroslysin or SEQ ID NO:8 chimeric or fusion proteins. The term “chimeric protein” or “fusion protein”, as used herein, comprises a Mucroslysin polypeptide or SEQ ID NO:8 polypeptide operatively linked to a non-Mucroslysin or SEQ ID NO:8 polypeptide. A “Mucroslysin polypeptide” or “SEQ ID NO:8 polypeptide”, as that term is used herein, refers to a polypeptide having an amino acid sequence corresponding to Mucroslysin or SEQ ID NO:8 protein or portions thereof, whereas “non-Mucroslysin or SEQ ID NO:8 polypeptide” refers to a polypeptide having an amino acid sequence corresponding to a protein which is not substantially identical to the Mucroslysin or SEQ ID NO:8 protein, i.e. a protein that is different from the Mucroslysin or SEQ ID NO:8 protein and is derived from the same or different organism. Within a Mucroslysin or SEQ ID NO:8 fusion protein, the Mucroslysin or SEQ ID NO:8 polypeptide can correspond to all or a portion of a Mucroslysin or SEQ ID NO:8 protein, preferably at least one biologically active portion of a Mucroslysin or SEQ ID NO:8 protein. Within the fusion protein, the term “operatively linked” is intended to indicate that the Mucroslysin or SEQ ID NO:8 polypeptide and the non-Mucroslysin or SEQ ID NO:8 polypeptide are fused in frame to each other. The non-Mucroslysin or SEQ ID NO:8 polypeptide can be fused to the N-terminus or C-terminus of the Mucroslysin or SEQ ID NO:8 polypeptide.

One useful fusion protein is a GST-Mucroslysin or SEQ ID NO:8 fusion protein in which the Mucroslysin or SEQ ID NO:8 sequences are fused to the C-terminus of the GST sequences. Such fusion proteins can facilitate the purification of recombinant Mucroslysin or SEQ ID NO:8.

In another embodiment, the fusion protein contains a signal sequence from another protein. In certain host cells (e.g. mammalian host cells), expression and/or secretion of Mucroslysin or SEQ ID NO:8 can be increased through the use of a heterologous signal sequence. For example, the gp67 secretory sequence of the baculovirus envelope protein can be used as a heterologous signal sequence (Current Protocols in Molecular Biology, Ausubel et al., eds., John Wiley & Sons, 1992). Other example of eukaryotic heterologous signal sequences include the secretory sequences of melittin and human placental alkaline phosphatase (Stratagene; La Jolla, Calif.) In yet another example, useful prokaryotic heterologous signal sequences include the phoA secretory signal (Molecular cloning, Sambrook et al, second edition Cold Spring Harbor Laboratory Press, 1989) and the protein A secretory signal (Pharmacia Biotech; Piscataway, N.J.).

Preferably, a Mucroslysin or SEQ ID NO:8 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in cohesive ends as appropriate, alkaline phosphatase treatment to avoid undesirable joining, and enzymatic ligation. In another embodiment, the fusion gene can by synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (E.g., a GST polypeptide). A Mucroslysin or SEQ ID NO:8-encoding nucleic acid can be cloned into such an expression vector such that the fusion moiety is linked in-frame to the Mucroslysin or SEQ ID NO:8-protein.

The present invention also pertains to variants of the Mucroslysin or SEQ ID NO:8 proteins which function as either Mucroslysin or SEQ ID NO:8 agonists or as Mucroslysin or SEQ ID NO:8 antagonists. Variants of the Mucroslysin protein can be generated by mutagenesis, e.g., discrete point mutation or truncation of the Mucroslysin or SEQ ID NO:8 protein. An antagonist of the Mucroslysin or SEQ ID NO:8 protein can inhibit one or more of the activities of the naturally occurring form of the Mucroslysin or SEQ ID NO:8 protein by, for example, competitively binding to a downstream or upstream member of a cellular signaling cascade which includes the Mucroslysin or SEQ ID NO:8 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the Mucroslysin or SEQ ID NO:8 protein.

Also, an isolated Mucroslysin or SEQ ID NO:8 protein or a portion or fragment thereof, can be used as an immunogen to generate antibodies that bind Mucroslysin or SEQ ID NO:8 using standard techniques for polyclonal and monoclonal antibody preparation. The full-length Mucroslysin or SEQ ID NO:8 protein can be used or, alternatively, the invention provides antigenic peptide fragments of Mucroslysin or SEQ ID NO:8 for use as immunogens. The antigenic peptide of Mucroslysin comprises at least 8 (preferably 10, 15, 20, 30, 40, 50 or more) amino acid residues of amino acid sequence shown in SEQ ID NO: 2, and encompasses an epitope of Mucroslysin such that an antibody raised against the peptide forms a specific immune complex with Mucroslysin. The antigenic peptide of SEQ ID NO:8 comprises at least 8 (preferably 10, 15, 20, 30, 40, 50 or more) amino acid residues of amino acid sequence shown in SEQ ID NO: 8, and encompasses an epitope of SEQ ID NO:8 protein such that an antibody raised against the peptide forms a specific immune complex with SEQ ID NO:8 protein.

A Mucroslysin or SEQ ID NO:8 immunogen typically is used to prepare antibodies by immunizing a suitable subject, (e.g., rabbit, goat, mouse or other mammal) with the immunogen. An appropriate immunogenic preparation can contain, for example, recombinantly expressed Mucroslysin or SEQ ID NO:8 protein or a chemically synthesized Mucroslysin or SEQ ID NO:8 polypeptide. The preparation can further include an adjuvant, such as Freund's complete or incomplete adjuvant, or similar immunostimulatory agent. Immunization of a suitable subject with an immunogenic Mucroslysin or SEQ ID NO:8 reparation induces a polyclonal anti-Mucroslysin or SEQ ID NO:8 antibody response.

Accordingly, another aspect of the invention pertains to anti-Mucroslysin or SEQ ID NO:8 antibodies. The term “antibody” as used herein refers to immunoglobulin molecules and immunologically active portions of immunoglobulin molecules, i.e., molecules that contain an antigen-binding site, which specifically binds an antigen, such as Mucroslysin or SEQ ID NO:8. A molecule that specifically binds Mucroslysin or SEQ ID NO:8 is a molecule which binds Mucroslysin or SEQ ID NO:8 but does not substantially bind other molecules in a sample, e.g., a biological sample, which naturally contains Mucroslysin or SEQ ID NO:8. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind Mucroslysin or SEQ ID NO:8. The term “monoclonal antibody” or “monoclonal antibody composition” refers to a population of antibody molecules that contain only one species of an antigen-binding site capable of immunoreacting with a particular epitope of Mucroslysin or SEQ ID NO:8. A monoclonal antibody composition thus typically displays a single binding affinity for a particular Mucroslysin or SEQ ID NO:8 protein with which it immunoreacts.

Polyclonal antibodies can be prepared as described above by immunizing a suitable subject with a Mucroslysin or SEQ ID NO:8 immunogen. The anti-Mucroslysin or SEQ ID NO:8 antibody titer in the immunized subject can be monitored over time by standard techniques, such as with an enzyme linked immunosorbent assay (ELISA) using immobilized Mucroslysin or SEQ ID NO:8. If desired, the antibody molecules directed against Mucroslysin or SEQ ID NO:8 can be isolated from the mammal (e.g., from the blood) and further purified by well-known techniques, such as protein A chromatography to obtain the IgG fraction. At an appropriate time after immunization, e.g., when the anti-Mucroslysin or SEQ ID NO:8 antibody titers are the highest, antibody-producing cells can be obtained from the subject and used to prepare monoclonal antibodies by standard techniques, such as the hybridoma technique originally described by Kohler and Milstein, Nature 256, 495-497, 1975, the human B cell hybridoma technique (Kozbor et al., Immunol Today 4, 72, 1983), the EBV-hybridoma technique (Cole et al., Monoclonal Antibodies and Cancer Therapy, Alan R. Liss, Inc., 77-96, 1985) or trioma techniques. The technology for producing various antibodies' monoclonal antibody hybridoma is well known (see generally Current Protocols in Immunology Coligan et al. (eds.) John Wiley & Sons, Inc., New York, N.Y., 1994). Briefly, an immortal cell line, typically a myeloma, is fused to lymphocytes, typically splenocytes, from a mammal immunized with a Mucroslysin or SEQ ID NO:8 immunogen as described above, and the culture supernatants of the resulting hybridoma cells are screened to identify a hybridoma producing a monoclonal antibody that binds Mucroslysin or SEQ ID NO:8.

Any of the many well known protocols used for fusing lymphocytes and immortalized cell lines can be applied for the purpose of generating an anti-Mucroslysin or SEQ ID NO:8 monoclonal antibody (see, e.g., Current Protocols in Immunology, supra; Galfre et al., Nature 266, 55052, 1997; R. H. Kenneth, in Monoclonal Antibodies: A new Dimension in biological Analyses, Plenum Publishing Corp., New York, N.Y., 1980; and Lerner, Yale J. Biol. Med. 54, 387-402, 1981. Moreover, one of ordinary skill will appreciate that there are many variations of such method, which also would be useful. Typically, the immortal cell line, such as a myeloma cell line, is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-AG4-1, P3-x63-Ag8.653 or Sp2/O—Ag14 myeloma lines. These myeloma lines are available from ATCC. Typically, HAT sensitive mouse myeloma cells are fused to mouse splenocytes using polyethylene glycol (“PEG”). Hybridomas resulting from the fusion are then selected using HAT medium, which kills unfused and unproductively fused myeloma cells (unfused splenocytes die after several days because they are not transformed). Hybridoma cells producing a monoclonal antibody of the invention are detected by screening hybridoma culture supernatants for antibodies that bind Mucroslysin using a standard ELISA assay.

Alternative to preparing monoclonal antibody-secreting hybridomas, a monoclonal anti-Mucroslysin or SEQ ID NO:8 antibody can be identified and isolated by screening recombinant combinatorial immunoglobulin library (e.g. an antibody phage display library) with Mucroslysin or SEQ ID NO:8 to thereby isolate immunoglobulin library member that bind Mucroslysin or SEQ ID NO:8. Kits for generating and screening phage display libraries are commercially available (e.g., the Pharmacia Recombinant Phage Antibody System Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication NO.

WO 93/01288; PCT Publication No. WO 92/01047; PCT Publication No. WO 92/09690; PCT Publication No. WO 90/02809; Fuchs et al., Bio/Technology 9, 1370-1372, 1991; Hay et al., Hum. Antibody Hybridomas 3, 81-85, 1992; Huse et al., Science 26, 1275-1281, 1989; Griffiths et al., EMBO J. 12, 725-734, 1993.

Additionally, recombinant anti-Mucroslysin or SEQ ID NO:8 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application; European Patent Application 173,494; PCT Publication No. WO 86/01533; U.S. Pat. No. 4,816,567; European Patent Application 125,023; Better et al., Science 249, 1041-1043, 1988; Liu et al., Proc. Natl. Acad. Sci. USA 84, 3439-3443, 1987; Liu et al., J. Immunol. 139, 3521-3526, 1987; Sun et al., Proc. Natl. Acad. Sci. USA 84, 214-218, 1987; Nishimura et al., Canc. Res. 47, 999-1005, 1987; Wood et al., Nature 314, 446-449, 1985; and Shaw et al., J. Natl. Cancer Inst. 80, 1553-1559, 1988; Morrison, Science 229, 1202-1207, 1985; Oi et al., Bio/Techniques 4, 214, 1986; U.S. Pat. No. 5,225,539; Jones et al., Nature 321, 552-525, 1986; Verhoeyan et al., Science 239, 1534, 1988; and Beidler et al., J Immunol. 141, 4053-4060, 1988.

An anti-Mucroslysin or SEQ ID NO:8 antibody (e.g., monoclonal antibody) can be used to isolate Mucroslysin or SEQ ID NO:8 by standard techniques, such as affinity chromatography or immunoprecipitation. An anti-Mucroslysin or SEQ ID NO:8 antibody can facilitate the purification of natural Mucroslysin or SEQ ID NO:8 from cells and of recombinantly produced Mucroslysin or SEQ ID NO:8 expressed in host cells. Moreover, an anti-Mucroslysin or SEQ ID NO:8 antibody can be used to detect Mucroslysin or SEQ ID NO:8 protein (e.g., in a cellular lysate or cell supernatant) in order to evaluate the abundance and pattern of expression of the Mucroslysin or SEQ ID NO:8 protein. Anti-Mucroslysin or SEQ ID NO:8 antibodies can be used diagnostically to monitor protein levels in tissue as part of a clinical testing procedure, e.g., to, for example, determine the efficacy of a given treatment regiment. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin and examples of suitable radioactive material include ¹²⁵I, ¹³¹I, ³⁵S or ³H.

III. Recombinant Expression Vectors and Host Cells

Another aspect of the invention relates to vectors, preferably expression vectors that contain a nucleic acid encoding Mucroslysin or fibrinlysin or a portion thereof. As used herein, the term “vector” refers to a nucleic acid molecule capable of transporting another nucleic acid to which it has been linked. One type of vector is a “plasmid”, which refers to a circular double stranded DNA loop into which additional DNA sequence can be ligated. Another type of vector is a viral vector to which additional DNA segments can be ligated and added into the viral genome. Certain vectors are capable of autonomous replication in a host cell into which they are introduced (e.g., bacterial vectors having a bacterial original of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described for example, in Goeddel, Gene Expression Technology: Methods in Enzymology 185, Academic Press, San Diego, Calif., 1990. Regulatory sequences include those, which direct constitutive expression of a nucleotide sequence in many types of host cell, and those, which direct expression of the nucleotide sequence only in certain host cells such as a tissue specific regulatory sequences. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g. Mucroslysin or fibrinlysin proteins, mutant forms of Mucroslysin or fibrinlysin, fusion proteins etc.). The recombinant expression vectors of the invention can be designed to express Mucroslysin or fibrinlysin in prokaryotic or eukaryotic cells, e.g., bacterial cells such as E. coli or insect cells (using baculovirus expression vectors), yeast cells, or mammalian cells. Suitable host cells are discussed further in Goeddel, Gene Expression Technology: Methods in Enzymology 185, Academic Press, San Diego, Calif., 1990. Alternatively, the recombinant expression vector can be transcribed and translated in vitro, for example, using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of recombinant protein; and 3) to aid in the purification of the recombinant protein by acting as a ligand in affinity purification. Often in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc., Smith and Johnson, Gene 67, 31-40, 1988), pMAL (New England Biolabs, Beverly, Mass.) and pRIT5 (Pharmacia Piscataway, N.J.) which fuse glutathione S-transferase (GST), maltose E binding protein, or protein A respectively to the target recombinant protein.

Examples of suitable inducible non-fusion E. coli expression vectors include pTrc (Amann et al., Gene 69, 301-315, 1988) and pET 11d (Studier et al., Gene Expression Technology: Methods in Enzymology 185, Academic Press, San Diego Calif., 60-89, 1990). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS 174 (DE3) from a resident X prophage harboring a T7 gn1 gene under the transcriptional control of the lacUV5 promoter.

One strategy to maximize recombinant protein expression in E. coli is to express the protein in a host bacterium with an impaired capacity to proteolytically cleave the recombinant protein (Gottesman, Gene Expression Technology: Methods in Enzymology 185, Academic Press, San Diego, Calif., 119-128, 1990). Another strategy is to alter the nucleic acid sequence of the nucleic acid to be inserted into an expression vector so that the individual codons for each amino acid are those preferentially utilized in E. coli (Wada et al., Nucleic Acids Res. 20, 2111-2118, 1992). Such alteration of nucleic acid sequence of the invention can be carried out by standard DNA synthesis techniques.

In another embodiment, the Mucroslysin or fibrinlysin expression vector is a yeast expression vector. Examples of vectors for expression in yeast S. cerevisiae include pYepSec1 (Baldari et al., EMBO J. 6, 229-234, 1987), pMFa (Kurjan and Herskowitz, Cell 30, 933-943, 1982), pJRY88 (Schultz et al., Gene 54, 113-123, 1987), pYES2 (Invitrogen Corporation, San Diego, Calif.), and picZ (Invitrogen Corporation, Sand Diego, Calif.).

Alternatively, Mucroslysin or fibrinlysin can be expressed in insect cells using baculovirus expression vectors. Baculovirus vectors available for expression of proteins in cultured insect cells (e.g., Sf 9 cells) include the pAc series (Smith et al, Mol. Cell Biol. 3, 2156-2165, 1983) and the pVL series (Lucklow and Summers, Virology 170, 31-39, 1989).

In yet another embodiment, a nucleic acid of the invention is expressed in mammalian cells using a mammalian expression vector. Examples of mammalian expression vectors include pCDM8 (Seed, Nature 329, 840, 1987) and pMT2PC (Kaufman et al., EMBO J. 6, 187-195, 1987). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapter 16 and 17 of Sambrook et al. (supra).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinker et al., Genes Dev. 1, 268-277, 1987), lymphoic-specific promoters (Calame and Eaton, Adv. Immunol. 43, 235-275, 1988), in particular promoters of T cell receptors (Winoto and Baltimore, EMBO J. 8, 729-733, 1989) and immunoglobulins (Banerji et al., Cell 33, 729-740, 1983; Queen and Baltimore, Cell 33, 741-748, 1983), neuron-specific promoters (e.g., the neurofilament promoter; Byrno and Ruddle, Proc. Natl. Acad. Sci. USA 86, 5473-5477, 1989), pancrease-specific promoters (Edlund et al., Science 230, 912-916, 1985) and mammary gland-specific promoters (e.g., milk whey promoter; U.S. Pat. No. 264,166). Developmentally regulated promoters are also encompassed, for example the murine hox promoters (Kessel and Gruss, Science 249, 374-379, 1990) and the α-fetoprotein promoter (Campes and Tighman, Gene Dev. 3, 537-546, 1989).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to Mucroslysin or fibrinlysin mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (Reviews—Trends in Geneticsm 1(1), 1986).

Another aspect of the invention relates to host cells into which a recombinant expression vector of the invention has been introduced. The term “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host call can be any prokaryotic or eukaryotic cell. For example, Mucroslysin protein can be expressed in bacterial cells such as E. coli, insect cells, yeast mammalian cells (such as Chinese hamster ovary cell (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art recognized techniques for introducing foreign nucleic acid (e.g., DNA) into a host cell, including calcium phosphate or calcium chloride co-precipitation, DEAE-dextran-mediate transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those, which confer resistance to drugs, such as G418, hygromycin and methotrexate. Nucleic acid encoding a selectable marker can be introduced into a host cell on the same vector as that encoding Mucroslysin or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce, i.e. express, a Mucroslysin or fibrinlysin protein. Accordingly, the invention further provides methods for producing Mucroslysin or fibrinlysin protein using the host cells of the invention. In one embodiment, a host cell of the invention (into which a recombinant expression vector encoding Mucroslysin or fibrinlysin has been introduced) in a suitable medium such that Mucroslysin or fibrinlysin protein is produced. In another experiment, the method further comprises isolating Mucroslysin or fibrinlysin from the medium or the host cell.

The host cells of the invention can also be used to produce nonhuman transgenic animals. For example, in one embodiment, a host cell of the invention is a fertilized oocyte or an embryonic stem cell into which Mucroslysin or fibrinlysin-encoding sequences have been introduced. Such host cells can then be used to create non-human transgenic animals in which exogenous Mucroslysin or fibrinlysin sequences have been introduced into their genome or homologous recombinant animals in which endogenous Mucroslysin or fibrinlysin sequences have been altered. Such animals are useful for studying the function and/or activity of Mucroslysin or fibrinlysin and for identifying and/or evaluating modulators of Mucroslysin or fibrinlysin activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, or preferably a mouse, in which an endogenous Mucroslysin or fibrinlysin gene has been altered by homologous recombination between the endogenous gene and an exogenous DNA molecule introduced into cell of the animal, e.g., an embryonic cell of the animal, prior to development of the animal.

A transgenic animal of the invention can be created by introducing Mucroslysin or fibrinlysin-encoding nucleic acid into the male pronuclei of a fertilized oocyte, e.g., by microinjection, retroviral infection, and allowing the oocyte to develop in a pseudopregnant female foster animal. The Mucroslysin or fibrinlysin cDNA sequence can be introduced as a transgene into the genome of a non-human animal. Alternatively, a nonhuman homologue of the human Mucroslysin or fibrinlysin gene can be isolated based on hybridization to the human Mucroslysin or fibrinlysin cDNA and used as a transgene. Intronic sequences and polyadenylation signals can also be included in the transgene to increase the efficiency of expression of the transgene. A tissue-specific regulatory sequence can be operably linked to the Mucroslysin or fibrinlysin transgene to direct expression of Mucroslysin or fibrinlysin protein to particular cells. Methods for generating transgenic animals via embryo manipulation and microinjection, particularly animals such as mice, have become conventional in the art and are described, for example, in U.S. Pat. No. 4,736,866, and 4,870,009, 4,873,191 and in Hogan, Manipulating the Mouse Embryo, (Cold Spring Harbor Laboratory Press, Colo. Spring Harbor, N.Y., 1986). Similar methods are used for production of other transgenic animals. A transgenic founder animal can be identified based upon the presence of the Mucroslysin or fibrinlysin transgene in its genome and/or expression of Mucroslysin or fibrinlysin mRNA in tissues of cells of the animals. A transgenic founder animal can then be used to breed additional animals carrying the transgene. Moreover, transgenic animals carrying a transgene encoding Mucroslysin or fibrinlysin can further be bred to other transgenic animals carrying other transgenes.

To create a homologous recombinant animal, a vector is prepared which contains at least a portion of a Mucroslysin or fibrinlysin gene (e.g., a human or a non-human homolog of the Mucroslysin gene) into which a deletion, addition or substitution has been introduced to thereby alter, e.g. functionally disrupt, the Mucroslysin or fibrinlysin gene. In a preferred embodiment, the vector is designed such that upon homologous recombination, the endogenous Mucroslysin or fibrinlysin gene is functionally disrupted (i.e. no longer encodes a functional protein; also referred to as a “knock out” vector). Alternatively, the vector can be designed such that, upon homologous recombination, the endogenous Mucroslysin or fibrinlysin gene is mutated or otherwise altered but still encodes the functional protein (e.g. the upstream regulatory region can be altered to thereby alter the expression of the endogenous Mucroslysin or fibrinlysin protein). In the homologous recombination vector, the altered portion of the Mucroslysin or fibrinlysin gene is flanked at its 5′ and 3′ ends by additional nucleic acid of the Mucroslysin or fibrinlysin gene to allow for homologous recombination to occur between the exogenous Mucroslysin or fibrinlysin gene carried by the vector and an endogenous Mucroslysin or fibrinlysin gene in an embryonic stem cell. The additional flanking Mucroslysin or fibrinlysin nucleic acid is of sufficient length for successful homologous recombination with the endogenous gene. Typically, several kilo bases of flanking DNA (both at the 5′ and 3′ ends) and included in the vector (see, e.g., Thomas and Capecch, Cell 51, 503, 1987 for a description of homologous recombination vectors). The vector is introduced into an embryonic stem cell line (e.g., by electroporation) and cells in which the introduced Mucroslysin or fibrinlysin gene has homologously recombined with the endogenous Mucroslysin or fibrinlysin gene are selected (see, e.g., Li et al., Cell 69, 915, 1992). The selected cells are then injected into a blastocyst of an animal (e.g., a mouse) to form aggregation chimera (see, e.g., Bradley in Teratocarcinomas and Embryonic Stem Cells: A Practical Approach, Robertson, ed. (IRL, Oxford, 113-152, 1987). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, Current Opinion in Bio/Technology 2, 823-829, 1991 and in PCT Publication Nos. WO 90/11354, WO 91/01140, WO 92/0968, and WO 93/04169.

In another embodiment, transgenic non-human animals can be produced which contain selected systems, which allow for regulated expression of the transgene. One example of such a system is the cre/loxP recombinase system of bacteriophage P1. For a description of the cre/losP recombinase system, see, e.g., Lakso et al., Proc. Natl. Acad. Sci. USA 89, 6232-6236, 1992. Another example of a recombinase system is the FLP recombinase system of Saccharomyces cerevisiae (O'Gorman et al., Science 251, 1351-1355, 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

IV. Screening Assays

The nucleic acid molecules, proteins, protein homologues, and antibodies described herein can be used in screening assays.

The invention provides a method (also referred to herein as a “screening assay”) for identifying modulators, i.e., candidate or test compounds or agents (e.g., peptides, peptidomimetics, small molecules or other drugs) which bind to Mucroslysin or fibrinlysin proteins or have a stimulatory or inhibitory effect on, for example, Mucroslysin or fibrinlysin expression or Mucroslysin or fibrinlysin activity.

In one embodiment, the invention provides assays for screening candidate or test compounds, which bind to or modulate the activity of a Mucroslysin or fibrinlysin protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12, 145, 1997). Examples of methods for the synthesis of molecular libraries can be found in the art, for example in: DeWitt et al., Proc. Natl. Acad. Sci. U.S.A. 90, 6909, 1993; Erb et al., Proc. Natl. Acad. Sci. USA 91, 11422, 1994; Zuckermann et al., J. Med. Chem. 37, 2678, 1994; Cho et al., Science 261, 1303, 1993; Carrell et al., Angew. Chem. Int. Ed. Engl. 33, 2059, 1994; Carell et al., Angew. Chem. Int. Ed. Engl. 33, 2061, 1994; and Gallop et al., J. Med. Chem. 37, 1233, 1994.

Libraries of compounds may be presented in solution (e.g., Houghten, Bio/Techniques 13, 412-421, 1992), or on beads (Lam, Nature 354, 82-84, 1991), chips (Fodor, Nature 364, 555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA 89, 1865-1869, 1992) or on phage (Scott and Smith, Science 249, 386-390, 1990; Devlin, Science 249, 404-406, 1990; Cwirla et al., Proc. Natl. Acad. Sci. 87, 6378-6382, 1990; and Felici, J. Mol. Biol. 222, 301-310, 1991).

Determining the ability of the test compound to modulate the activity of Mucroslysin Or fibrinlysin or a biologically active portion thereof can be accomplished, for example, by determining the ability of the Mucroslysin or fibrinlysin protein to bind to or interact with a Mucroslysin or fibrinlysin target molecule. As used herein, a “target molecule” is a molecule with which a Mucroslysin or fibrinlysin protein binds or interacts in nature, for example, a molecule associated with the internal surface of a cell membrane or a cytoplasmic molecule. A Mucroslysin or fibrinlysin target molecule can be a non-Mucroslysin or fibrinlysin molecule or a Mucroslysin or fibrinlysin protein or polypeptide of the present invention. In one embodiment, a Mucroslysin or fibrinlysin target molecule is fibrinogen, which molecule contributes to the formation of occlusive thrombi.

Determining the ability of the Mucroslysin or fibrinlysin protein to bind to or interact with a Mucroslysin or fibrinlysin target molecule can be accomplished by one of the methods described above for determining direct binding. In a preferred embodiment, determining the ability of the Mucroslysin or fibrinlysin protein to bind to or interact with a Mucroslysin or fibrinlysin target molecule can be accomplished by determining the activity of the target molecule. For example, the activity of the target molecule can be determined detecting catalytic/enzymatic activity of the target in an appropriate substrate, e.g. analyzing the thrombolytic activity of the target molecule in artificial thrombi induced rats injected with Mucroslysin or fibrinlysin.

In yet another embodiment, an assay of the present invention is a cell-free assay comprising contacting a Mucroslysin or fibrinlysin protein or biologically active portion thereof with a test compound and determining the ability of the test compound to bind to the Mucroslysin or fibrinlysin protein or biologically active portion thereof. Binding of the test compound to the Mucroslysin or fibrinlysin protein can be determined either directly or indirectly as described above. In a preferred embodiment, the assay includes contacting the Mucroslysin or fibrinlysin protein or biologically active portion thereof with a known compound which binds Mucroslysin or fibrinlysin to form an assay mixture, contacting the assay mixture with a test compound, and determining the ability of the test compound to interact with a Mucroslysin or fibrinlysin protein, wherein determining the ability of the test compound to interact with a Mucroslysin or fibrinlysin protein comprises determining the ability of the test compound to preferentially bind to Mucroslysin or fibrinlysin or biologically active portion thereof as compared to the known compound.

In another embodiment, an assay is a cell-free assay comprising contacting Mucroslysin or fibrinlysin protein or biologically active portion thereof with a test compound and determining the ability of the test compound to modulate (e.g., stimulate or inhibit) the activity of the Mucroslysin or fibrinlysin protein or biologically active portion thereof. Determining the ability of the test compound to modulate the activity of Mucroslysin or fibrinlysin can be accomplished, for example, by determining the ability of the Mucroslysin or fibrinlysin protein to bind to a Mucroslysin or fibrinlysin target molecule by one of the methods described above for determining direct binding. In an alternative embodiment, determining the ability of the test compound to modulate the activity of Mucroslysin or fibrinlysin can be accomplished by determining the ability of the Mucroslysin or fibrinlysin protein further modulate a Mucroslysin or fibrinlysin target molecule. For example, the catalytic/enzymatic activity of the target molecule on an appropriate substrate can be determined as previously described.

In yet another embodiment, the cell-free assay comprises contacting the Mucroslysin or fibrinlysin protein or biologically active portion thereof with a known compound which binds Mucroslysin or fibrinlysin to form an assay mixture, contacting the assay mixture with a test compound, and determining the ability of the test compound to interact with a Mucroslysin or fibrinlysin protein, wherein determining the ability of the test compound to interact with a Mucroslysin or fibrinlysin protein comprises determining the ability of the Mucroslysin or fibrinlysin protein to preferentially bind to or modulate the activity of a Mucroslysin or fibrinlysin target molecule. The cell-free assays of the present invention are amenable to use of either the soluble form or the membrane-associated form of Mucroslysin or fibrinlysin. A membrane-associated form of Mucroslysin or fibrinlysin refers to Mucroslysin or fibrinlysin that interacts with a membrane-bound target molecule. In the case of cell-free assays comprising the membrane-associated form of Mucroslysin or fibrinlysin, it may be desirable to utilize a solubilizing agent such that the membrane-associated form of Mucroslysin or fibrinlysin is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton.RTM. X-100, Triton.RTM. X-114, Thesit.RTM., Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either Mucroslysin or fibrinlysin or its target molecule to facilitate separation of complexed from uncomplexed forms of one or both of the proteins, as well as to accommodate automation of the assay. Binding of a test compound to Mucroslysin or fibrinlysin, or interaction of Mucroslysin or fibrinlysin with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/Mucroslysin or fibrinlysin fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or Mucroslysin or fibrinlysin protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Mucroslysin or fibrinlysin binding or activity determined using standard techniques.

Other techniques for immobilizing proteins on matrices can also be used in the screening assays of the invention. For example, either Mucroslysin or fibrinlysin or its target molecule can be immobilized utilizing conjugation of biotin and streptavidin. Biotinylated Mucroslysin or fibrinlysin or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with Mucroslysin or fibrinlysin or target molecules but which do not interfere with binding of the Mucroslysin or fibrinlysin protein to its target molecule can be derivatized to the wells of the plate, and unbound target or Mucroslysin or fibrinlysin trapped in the wells by antibody conjugation. Methods for detecting such complexes, in addition to those described above for the GST-immobilized complexes, include immunodetection of complexes using antibodies reactive with the Mucroslysin or fibrinlysin or target molecule, as well as enzyme-linked assays which rely oil detecting an enzymatic activity associated with the Mucroslysin or fibrinlysin or target molecule.

In another embodiment, modulators of Mucroslysin or fibrinlysin expression are identified in a method in which a cell is contacted with a candidate compound and the expression of Mucroslysin or fibrinlysin mRNA or protein in the cell is determined. The level of expression of Mucroslysin or fibrinlysin mRNA or protein in the presence of the candidate compound is compared to the level of expression of Mucroslysin or fibrinlysin mRNA or protein in the absence of the candidate compound. The candidate compound can then be identified as a modulator of Mucroslysin or fibrinlysin expression based on this comparison. For example, when expression of Mucroslysin or fibrinlysin mRNA or protein is greater (statistically significantly greater) in the presence of the candidate compound than in its absence, the candidate compound is identified as a stimulator of Mucroslysin or fibrinlysin mRNA or protein expression. Alternatively, when expression of Mucroslysin or fibrinlysin mRNA or protein is less (statistically significantly less) in the presence of the candidate compound than in its absence, the candidate compound is identified as an inhibitor of Mucroslysin or fibrinlysin mRNA or protein expression. The level of Mucroslysin or fibrinlysin mRNA or protein expression in the cells can be determined by methods described herein for detecting Mucroslysin or fibrinlysin mRNA or protein.

In yet another aspect of the invention, the Mucroslysin or fibrinlysin proteins can be used as “bait proteins” in a two-hybrid assay or three hybrid assay (see, e.g., U.S. Pat. No. 5,283,317; Zervos et al., Cell 72, 223-232, 1993; Madura et al., J. Biol. Chem. 268, 12046-12054, 1993; Bartel et al., Bio/Techniques 14, 920-924, 1993; Iwabuchi et al., Oncogene 8, 1693-1696, 1993; and PCT Publication No. WO 94/10300), to identify other proteins, which bind to or interact with Mucroslysin or fibrinlysin (“Mucroslysin or fibrinlysin-binding proteins” or “Mucroslysin or fibrinlysin-bp”) and modulate Mucroslysin or fibrinlysin activity. Such Mucroslysin or fibrinlysin-binding proteins are also likely to be involved in structural formation of the Mucroslysin or fibrinlysin protein.

The two-hybrid system is based on the modular nature of most transcription factors, which consist of separable DNA-binding and activation domains. Briefly, the assay utilizes two different DNA constructs. In one construct, the gene that codes for Mucroslysin or fibrinlysin is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a Mucroslysin or fibrinlysin-dependent complex, the DNA-binding and activation domains of the transcription factor is brought into close proximity. This proximity allows transcription of a reporter gene (e.g., LacZ), which is operably linked to a transcriptional regulatory site responsive to the transcription factor. Expression of the reporter gene can be detected and cell colonies containing the functional transcription factor can be isolated and used to obtain the cloned gene that encodes the protein that interacts with Mucroslysin or fibrinlysin.

V. Pharmaceutical Compositions

The Mucroslysin or fibrinlysin nucleic acid molecules, Mucroslysin or fibrinlysin proteins, and anti-Mucroslysin or fibrinlysin antibodies (also referred to as “active compounds”) of the invention can be incorporated into pharmaceutical compositions suitable for administration. Such compositions typically comprise the nucleic acid molecule, protein, or antibody and a pharmaceutically acceptable carrier. The term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene, glycols, glycerine, propylene glycol or other synthetic solvents; antibacterial agents such as benzyl alcohol or methyl parabens; antioxidants such as ascorbic acid or sodium bisulfite: chelating agents such as ethylenediaminetetraacetatic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions a desterile powders for extemporaneous preparation of sterile injectable solutions or dispersion. For intravenous administration suitable carriers include physiological saline, baceteriostatic water, Cremophor EL™ (BASF: Parsippany, N.J.) or phosphate buffered saline (PBS). In all cases, the composition must be sterile and should be fluid to the extent that easy syringability exists. PH must be stable under the conditions of manufacture and storage must be preserved against the contaminating action of microorganisms such as bacteria and fungi. The carrier can be a solvent or dispersion medium containing water, ethanol polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipeints and used in the form of tablets, troches, or capsules. Oral compositions can also be prepared using fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum gragacanth or gelatin; an excipient such as starch or lactose, a disintegrating agent such as alginic acid Primogel, or corn starch; a lubricant such as magnesium stearate or sterotes; a glidant such as collodial silicon dioxide; a sweetening agent such as a sucrose or saccharin; or a flavoring agent such as peppermint, methyl salicylate, or orange flavoring. For administration by inhalation, the compounds are delivered in the form of an aerosol spray form pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally well known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories or retention enemas for rectal delivery.

VI. Methods of use

Another aspect of the invention relates to methods of using the Mucroslysin or fibrinlysin related protein, e.g. proteins encoded by SEQ ID NO:2, SEQ ID NO:4, or SEQ ID NO:8 to induce fibrinogenolytic activity. The method of the invention can be practiced in vitro or in vivo. In the in vitro situation, one of said proteins is delivered to, e.g., mixing into, an in vitro environment, for example, a mixture of solutions containing fibrinogen. Said protein interacts with fibrinogen in the in vitro environment to lyse the proteins by cleaving off the α, β, and γ chain of the fibrinogen protein. In the in vivo situation, said protein is delivered, e.g. via various routes of administration such as by injection, oral intake, dermal application etc., into a living being, for example, human, rat, mice, canine, and rabbit etc. Said protein induces thrombolysis resulting from fibrinogenolytic act of the protein. Furthermore, the in vivo fibrinogenolytic activity of said proteins is devoid of any hemorrahagic side effect.

### EXAMPLES

### Example 1

Isolation and Characterization of SEQ ID NO:7

cDNA Library Construction and Screening

Ten micrograms of poly(A)⁺ RNA isolated from the adult T. mucrosquamatus venom gland were used to prepare double-stranded cDNA using the method described by Sambrook et al., Molecular Cloning: a Laboratory Manual, 8.3-8.8, 1989. Venom gland cDNA libraries were constructed in a λgt11 cloning system (Promega). The cDNA library, which contains approximately 1.0×10⁵ plaques, was initially screened using anti-triflavin polyclonal antisera. Hybridization was carried out at 37° C. for 16 hours in a mixture containing 10× Denhardt solution, 6×SSC, 50% formamide, and 100 mg/ml sonicated salmon sperm DNA with a [¹²⁵I] labeled protein A secondary antibody. The anti-triflavin antibody was synthesized using triflavin antigen, a purified component isolated from T. flavoviridis venom.

5′-Rapid Amplification of cDNA Ends (5′-RACE)

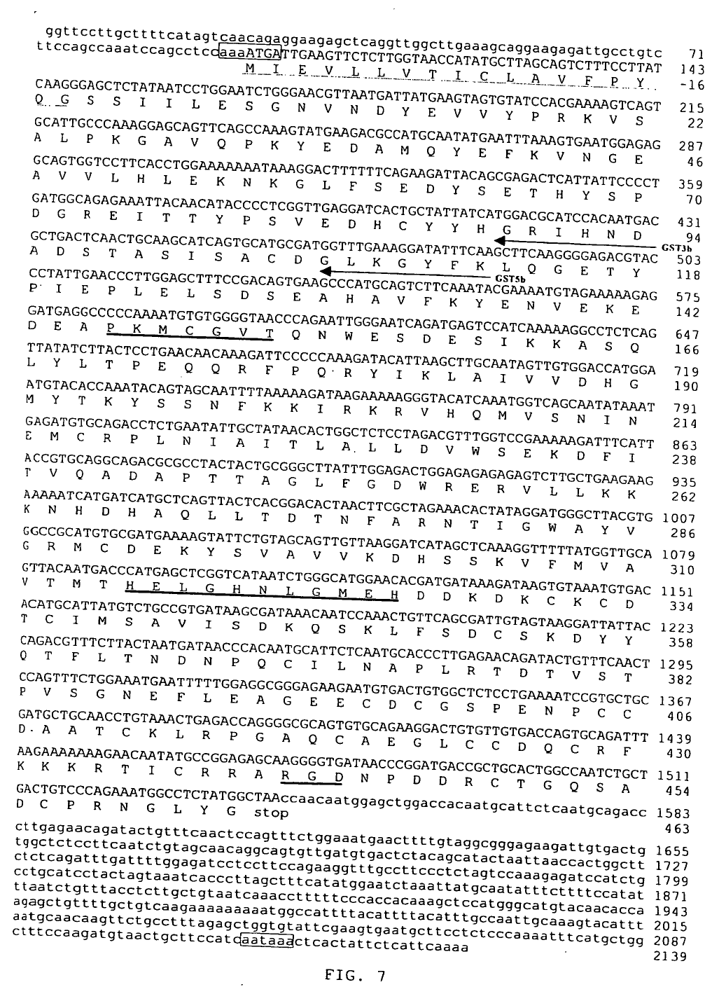

Two specific antisense primers were synthesized according to the 5′-end sequence of the partial SEQ ID NO:7 cDNA isolated from the snake venom cDNA library. These were the GST5b primer: 5′-TAT TTG AAG ACT GCA TGG GC-3′ and a nested GST3b primer: 5′-GGT ACG TCT CCC CTT GAA GC-3′ for 5′-RACE (FIG. 7). Synthesis of the 5′-end cDNA of SEQ ID NO:7 was performed using a 5′-RACE System kit according to the manufacturer protocols (Life Technologies, NY, U.S.A.). The first complementary strand was synthesized by reverse transcriptase using the GST5b primer, and the product was extended at the 3′-end with oligo d(C) with terminal transferase. The 5′-end region of the full-length cDNA was then amplified by means of a polymerase chain reaction (PCR) using the nested GST3b primer and an anchor primer complementary to the oligo d(C). The reaction was subjected to 39 cycles of heat denaturation at 94° C. for 60 sec, primer annealing at 51° C. for 40 sec, and primer extension at 72° C. for 5 min with UlTma polymerase (Perkin Elmer, Calif., U.S.A.) using a 1605 Air Thermo-Cycler (Idaho Technology, ID, U.S.A.).

DNA Sequencing, Homology Search and Sequence Comparison

SEQ ID NO:7 cDNA was subcloned into M13 mp18/19 bacteriophage and sequenced using the dideoxy chain termination method previously described by Sanger (Sanger, F., Nicklen S. & Coulson, A. R., Proc. Natl. Acad. Sci. USA. 74, 5463-5467, 1977.). Comparisons of the nucleotide sequences and deduced amino acid sequences were performed using the sequence analysis software package provided by GCG (Genetics Computer Group, Inc., WI, U.S.A.).

RNA Preparation and Analysis

Organs of T. mucrosquamatus were surgically removed and immediately frozen in liquid nitrogen before being ground into a powder form. The total RNA was extracted from the venom gland, brain, lung, testis, liver, and heart of T. mucrosquamatus using the guanidine isothiocyanate method previously described (Chirgwin et al., Biochemistry 18, 5294-5299, 1979.). The cDNA probes were labeled with [α-³²P]dCTP (Amersham, Ill., U.S.A.) using a rediprimer DNA labeling system (Amersham, Ill., U.S.A.). Northern blot hybridization was performed with the labeled SEQ ID NO:7 cDNA as a probe at 42° C. for 16 hours.

Result

The SEQ ID NO:7 cDNA was isolated by screening 1.0×10⁵ recombinant clones with an anti-triflavin antisera. Twenty immunopositive clones containing SEQ ID NO:7 cDNA were obtained using a [¹²⁵I] labeled protein-A antibody screening method. Analysis of 20 positive clones indicated that the DNA insert ranged in size from 0.7 to 1.4 kilobase pairs (Kb), none of which were full-length. A 1.4 Kb clone was selected and used to obtain a full-length cDNA. As a result, an additional 0.67 Kb DNA 5′ of the 1.4 Kb sequence was obtained by the method of 5′-rapid amplification of cDNA ends (RACE) (data not shown). The nucleotide sequence and deduced amino acid sequence are shown in FIG. 7. The 2139 bp cDNA had an open reading frame starting at nucleotide 96 and ending with the termination codon, TAA, at position 1541. The first in-frame methionine codon was found to be located at position 96 and was contained within the translation initiation consensus sequence, AAAATGA (Kozak, M., J. Cell Biol. 115, 887-903, 1991). The coding region had 1475 bases and could code for 481 amino acids with a calculated molecular weight of approximately 52.1 kDa. The 5′-untranlated region started from nucleotides 1 to 95. The 3′-untranslated region contained a long stretch of 598 nucleotides starting from nucleotide 1542 and extending to 2139 nucleotide, with a polyadenylation signal, AATAAA, located 20 bp upstream from the poly(A)⁺ tail at position 2114 (FIG. 7).

Using the von Heijne method (von Heijne, G., Eur. J. Biochem. 133, 17-21, 1983.), a putative signal peptide of 18 amino acids was predicted from the N-terminal residues of SEQ ID NO:7 (FIG. 7). A sequence analysis was performed using the BLAST from the GCG software package. The homology of SEQ ID NO:7 with trimucrin of T. mucrosquamatus was 98% (Tsai et al., Biochim. Biophys. Acta. 1200, 337-340, 1994.), trigramin from T. gramineus 86% (Neeper, M. P. & Jacobson, M. A., Nucleic Acids Res. 18, 4255, 1990.), halystatin from Agkistrodon halys 83% (Fujisawa et al., Acta Neurochir Suppl. 60, 193-196, 1994.), MT-c from Agkistrodon halys brevicadus 80% (Jeon, 0. H. & Kim, D. S., Biochem. Mol. Biol. Int. 47, 417-425, 1999.), atrolysin e from C. atrox 78% (Shimokawa et al., Arch. Biochem. Biophys. 335, 283-294, 1996.), and lebetase from Macrovipera lebetina 75% (Trummal et al., Biochim. Biophys. Acta 1476, 331-336, 2000.), as can be seen in FIG. 8. This high homology suggested that these metalloproteinase genes all consist of a zymogen prodomain, a proteinase domain, and a disintegrin (or disintegrin-like) domain. There were 23 cysteine residues that were highly conserved between SEQ ID NO:7 and this family of metalloproteinases (FIG. 8).

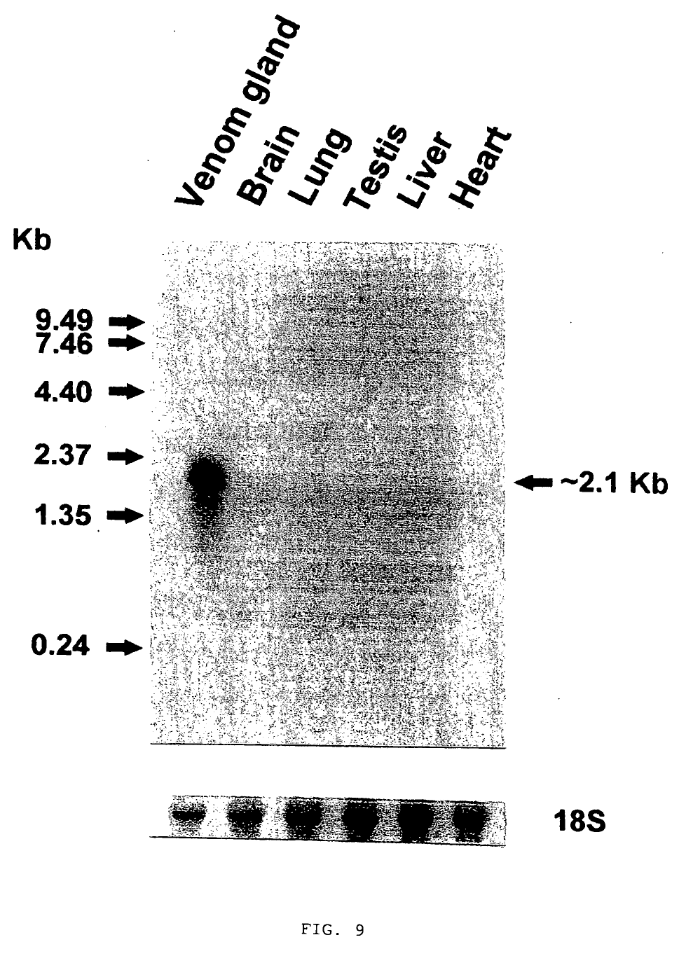

Northern blot analysis of SEQ ID NO:7 mRNA in six major anatomic organs: venom gland, brain, lung, testis, liver, and the heart from T. mucrosquamatus revealed a tissue-specific hybridization product of 2.1 Kb exclusively in the venomous gland (FIG. 9). Northern blot analysis indicated the tissue-specific expression of the SEQ ID NO:7 gene in the venom gland as well as its corresponding length of the cDNA.

### Example 2

Isolation of SEQ ID NO:3

Isolation and Amplification of SEQ ID NO: 3

In order to isolate the cDNA region as shown in SEQ ID NO:3 from the cDNA of SEQ ID NO:7, SEQ ID NO:3 is being specifically targeted and amplified in the PCR process using two designed primers. A 5′ primer was designed and contains a BamHI restriction site. The 5′ primer has the sequence of 5′-CCG GAT CCG AAC AAC AAA GAT TCC CCC AAA-3′, with the underlined portion denoting enzyme restriction site. A 3′ primer was also designed and contains an EcoRI restriction enzyme. The 3′ primer has the sequence of 5′-CGA ATT CGC GGG TGC ATT GAG AAT GCA TTG-3′, with the underlined portion denoting the enzyme restriction site. The above two primers were used in a PCR reaction to selectively amplify SEQ ID NO:3.

### Example 3

Construction and Expression of Mucroslysin Expression Plasmid

Construction of Mucroslysin Expression Plasmid

The expression plasmid was constructed by ligating SEQ ID NO:1 cDNA into a pET21 a vector (Novagen Inc., WI, U.S.A.) at the BamHI and EcoRI sites. Plasmid containing a 1.47 Kb DNA insert, designated as pMucroslysin, was then introduced into E. coli BL21 (DE3) cells for protein expression.

Expression and Purification of Fusion Protein

BL21(DE3) cells containing pMucroslysin were grown to a late log phase (A₆₀₀=0.3˜0.4) in Luria-Bertani broth and induced with 1 mM isopropyl-β-D-thiogalactoside (IPTG) for 2 hr. The cells were pelletted and sonicated in an 8M urea buffer (8M urea, 0.1 M NaH₂PO₄, 10 mM Tris-HCl pH8.0). The resulting lysate containing the recombinant Mucroslysin protein was incubated with His-bind affinity resin at 4° C. for 2 hr. The resin was then washed twice with the 8M-urea buffer. The recombinant protein was eluted from the resin with the same buffer containing 400 mM of imidazole. The yield of the purified protein was 10 mg/liter of bacterial culture.

Western Blotting Analysis

Western blotting was performed according to the methods of Burnette (Burnette, W., Anal. Biochem. 112, 195-203, 1981.). The membrane was first treated with rabbit anti-T. mucrosquamatus venom antiserum (1:5000), then reacted with peroxidase-conjugated secondary antibody of goat anti-rabbit IgG. The protein was detected using 3,3′-diaminobenzidine (DAB) and hydrogen peroxide.

Antibody Preparation

Thirty-five μl of crude venom was extracted from T. mucrosquamatus and inactivated with an equal volume of 10% formalin at room temperature for 1 hr. Inactivated venom was diluted to a volume of 0.5 ml with PBS buffer (137 mM NaCl, 2.68 mM KCl, Na₂HPO₄ 10 mM, KH₂PO₄ 1.76 mM, pH 7.4), and then mixed thoroughly with 0.5 ml of Freund complete adjuvant. The mixture (1.0 ml) was then injected into a restrained rabbit with multiple intradermal applications. Immunization was further administered by means of boosting three times at 2- to 3-week intervals.

Result

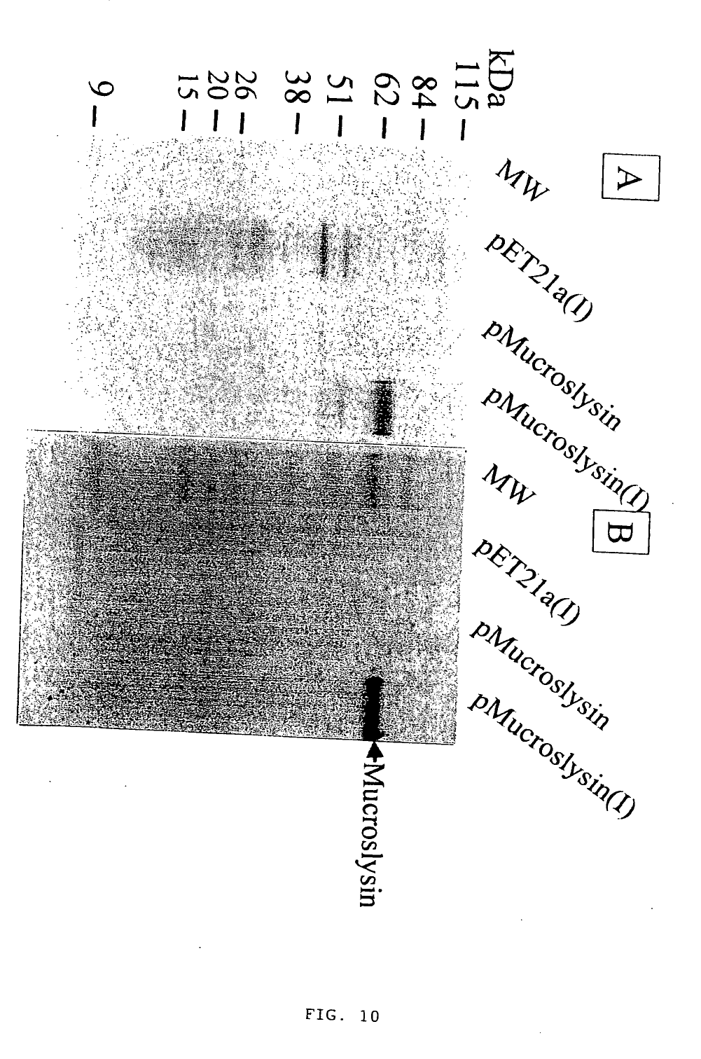

SEQ ID NO:1 was excised from the M13/mp18 vector using BamHI/EcoRI and subsequently cloned into the pET21a expression vector with an in-frame histidine tag down stream of the EcoRI cloning site. After IPTG induction, the expressed fusing protein was analyzed using both 10% tricine SDS-PAGE (FIG. 10) and Western blotting (FIG. 10). The Mucroslysin recombinant fusion protein, purified through a His-bind-resin, was recognized by polyclonal antibodies raised against the venom of T. mucrosquamatus and was found to have a molecular weight of 52 kDa.

### Example 4

Construction and Expression of SEQ ID NO:3 Expression Plasmid

Construction of SEQ ID NO:3 Expression Plasmid

The isolated SEQ ID NO:3 cDNA was amplified by PCR and subcloned into a pET32a(+) bacteria expression vector to produce the recombinant protein in E. coli. DNA sequencing of the PCR product showed that no substitutions were introduced by the reaction of amplification. The recombinant plasmid (pFibrinlysin) was used to transform E coli BL21(DE3) cells.

Expression and Purification of Fusion Protein

The transformed E. coli BL21(DE3) cells were induced with 1 mM IPTG to produce the recombinant protein. The induction with IPTG at 37° C. led to the production of a major fusion protein of 40 kDa in the cell lysate within 2 hrs, as shown in FIG. 1. The major expression protein was recovered as inclusion bodies in the pellet. Recombinant proteins were then isolated by affinity chromatography in a His-bind gel under denaturing conditions with urea. Proteins that bound to the resin were eluted with 400 mM imidazole buffer.

Western Blotting Analysis

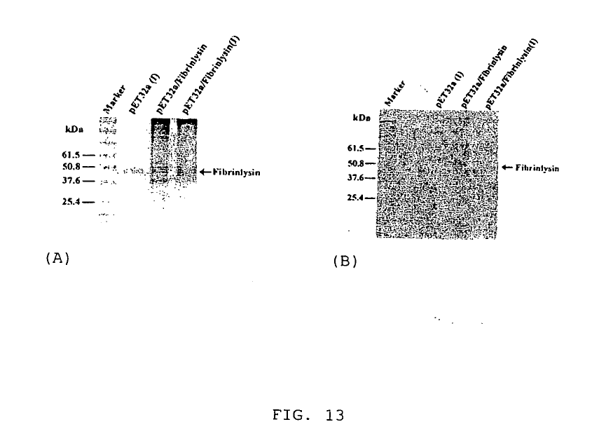

The purified proteins were analyzed by using both 10% tricine SDS-PAGE (FIG. 13A) and Western blotting (FIG. 13B). The rabbit anti-T. mucrosquamatus venom antiserum was diluted 5000 times for the western blot analysis. The recombinant expressed protein, detected as a major band in the gel and recognized by a polyclonal antibody raised against the venom of T. mucrosquamatus, was found to have a molecular weight of 40 kDa. At this step, the yield of the recombinant was 8 mg/liter.

### Example 5

In Vivo and In Vitro Analysis of Mucroslysin Protein

Refolding and Characterization of the Recombinant Protein Expressed in E. coli

Purified Mucroslysin protein was adjusted to a final concentration of 5 μg/ml protein.

Diluted protein was refolded in a refolding buffer (50 mM Tris-HCl, pH 9.0/0.1 mM EDTA/1 mM ZnCl₂/5 mM CaCl₂/2 mM reduced glutathione/0.2 mM oxidized glutathione/30% glycerol/0.01% Tween-20/0.3M urea) at 4° C. for 120 hrs. The refolded protein was dialyzed against the analytical buffer (50 mM Tris-HCl, pH 7.5/1 mM CaCl₂/1% glycerol/0.01% Tween-20) at 4° C. for another 24 hrs to remove the residual urea. The refolded protein was concentrated to 1 mg/ml with Centricon (Amicon, Beverly, Mass.) at 4° C. Functional analysis of refolded protein was carried out in 7.5 μl PBS containing 1 mM CaCl₂ and 10 μg fibrinogen at 37° C. for 0, 3, 6, 18, 24, 30, and 36 hrs, respectively. The protein ratio of fibrinogen to the refolded protein was 100/1 (w/w). Digestion reactions were stopped by heating the reaction mixtures at 100° C. for 5 min, and the samples were electrophoresed on SDS gels.

Thrombolysis Assay by Angiography

In vivo fibrinolytic activity of the recombinant protein was tested on artificial thrombi induced in the posterior vena cava of Sprague-Dawley (SD) rats. Thrombolysis was then analyzed by using angiographic techniques over a period of two hours. A catheter was first inserted in the right femoral vein of the anesthetized SD rats for the purpose of drug administration or blood sampling. Artificial thrombi were induced in the isolated posterior vena cava by injection of 15 μl of bovine fibrinogen solution (5%) and 5 μl of thrombin (100 u/ml) after laparotomy operation. Recombinant Mucroslysin protein at a dosage of 1.0-6.0 mg/kg was injected through a femoral vein catheter into the rats one hour after thrombus induction. Each angiograffin (Angiovist 370, Berlex Labs) in a volume of 0.5 ml was injected through the catheter right before taking the angiogram. Four angiograms were taken at time 0 min, 15 min, 30 min, and 120 min of the experiment (Willis et al., Thromb. Res. 53, 19-29, 1989).

Histological Examination of Animals Treated with Recombinant Mucroslysin Protein

Ten Sprague-Dawley rats were intravenously injected with recombinant Mucroslysin protein at a dosage of 10 mg/kg body weight. After 24 hr, tissue sections from the kidney, liver, heart and lung were fixed with 10% formalin and embedded in paraffin. Sections were stained with hematoxylin and eosin. Each tissue was examined for necrosis and hemorrhage.

Result

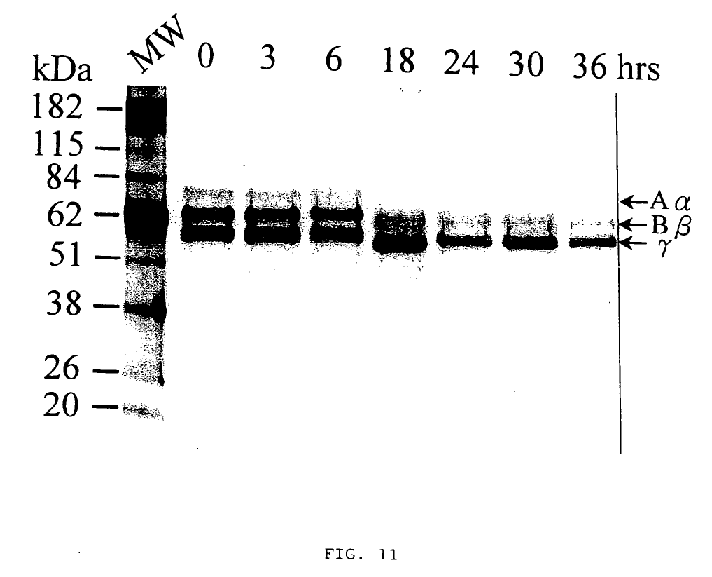

Purified recombinant Mucroslysin protein was refolded in a zinc and calcium ion-containing glutathione refolding buffer system. Proteolytic activities of the refolded proteins were tested by analyzing their ability to digest fibrinogen in vitro and their ability to lyse artificial thrombi in rats. In the fibrinogenolytic assay, refolded proteins were incubated with 10 μg of bovine fibrinogen for 0, 3, 6, 18, 24, 30, and 36 hours respectively, and the cleavage patterns were analyzed by using 10% SDS-PAGE. The Aα-chain of fibrinogen was completely digested at 18 hr after incubation. However, the digestion of the Bβ-chain began at 18 hr. The Bβ-chain was completely digested after 36 hrs. Mucroslysin protein also exhibited the ability to cleave the γ-chain of fibrinogen. Only hemorrhagic toxin f (HT-f) (Nikai et al., Arch. Biochem. Biophys. 231, 309-319, 1984.) and kistomin (Huang et al., Biochim. Biophys. Acta 1160, 262-268, 1992.) have been shown to have this activity. An extra band in the bottom of the gel at 30 kDa was accumulated, corresponding to the disappearance of Bβ-chain and γ-chain of fibrinogen (FIG. 11).

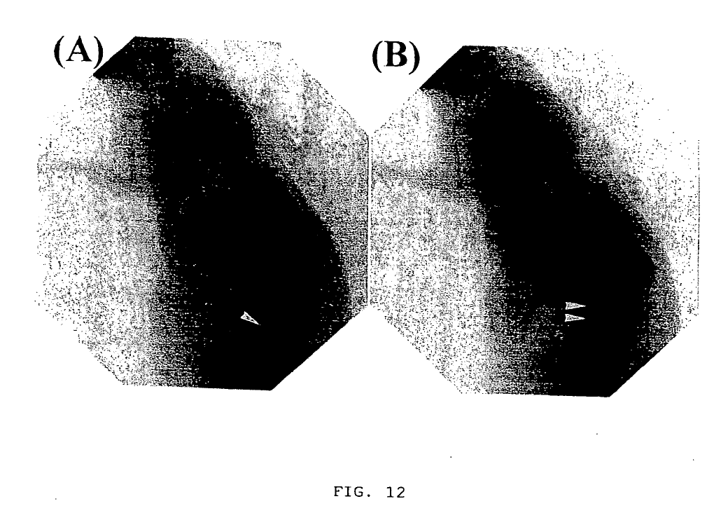

The thrombolytic activity of Mucroslysin protein in vivo was tested on artificial thrombi induced in the posterior vena cava of Sprague-Dawley rats (Willis et al., Thromb. Res. 53, 19-29, 1989.). Thrombolysis was then analyzed by angiographic techniques over a 2-hr period. Intravenous administration of the recombinant Mucroslysin protein, at a dosage of 1.0 mg/kg, resulted in thrombolysis by recanalization of the originally occluded vein within 15 min after the administration of the Mucroslysin protein. The thrombi was completely dissolved within two hours (FIG. 12). Histological examination of kidney, liver, heart and lung tissue showed neither necrosis nor hemorrhage (data not shown).

### Example 6

In Vivo and In Vitro Analysis of SEQ ID NO:3 Protein

Refolding and Characterization of the Recombinant Protein Expressed in E. coli

To reconstitute the biological activity of the expressed recombinant protein in vitro, purified recombinant fibrinlysin protein was refolded in a zinc and calcium ion-containing glutathione refolding buffer system. Proteolytic activities of the refolded proteins were tested by analyzing their ability to digest fibrinogen. Proteolytic activities were analyzed by incubating the refolded proteins with 10 μg bovine fibrinogen. Incubation times are indicated on the top of the figures and the cleavage patterns were analyzed on a 10% SDS-PAGE. The Aα-, Bβ-, and γ-chain of fibrinogen are indicated on the right. The protein ladder markers are indicated on the left (25.4-61.5 kDa).

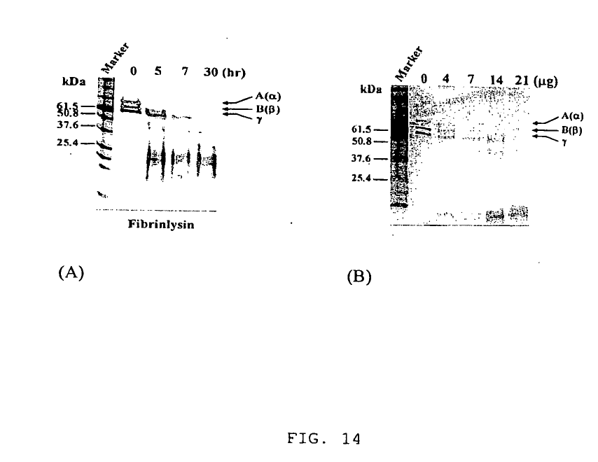

In the time-dependent fibrinogenolytic assay in vitro, the Aα-chain of fibrinogen was completely digested at 5 hr after incubation. The Bβ-chain was completely digested after 7 hrs. Fibrinlysin also exhibited the ability to cleave the γ-chain of fibrinogen and the γ-chain was completely digested after 30 hrs. Four to five extra bands appeared in the bottom of the gel at 45, 30, 25, 20; and 15 kDa accumulated corresponding to the disappearance of Aα-, Bβ-, and γ-chain of fibrinogen (FIG. 14B). Only hemorrhagic toxin f (HT-f) (Nikai, et al., 1984) and kistomin (Huang, et al., 1992) have been shown to have this activity. In the dose-dependent fibrinogenolytic assay in vitro, the Aα-chain of fibrinogen was completely digested with 4 μg of the refolded protein. The Bβ-chain was completely digested with 7 μg of the refolded protein. The γ-chain also was completely digested with 21 μg of the refolded protein (FIG. 14A). Several extra bands appeared in the bottom of the gel at 45, and molecular weights lower than 10 kDa were accumulated corresponding to the disappearance of Aα-, Bβ-, and γ-chain of fibrinogen (FIG. 14A).

Morphological Examination of Animals Treated with Recombinant Fibrinlysin Protein

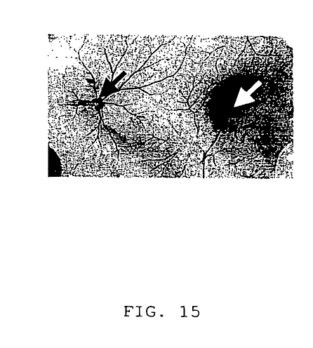

The hemorrhagic activity of fibrinlysin protein was tested in vivo on the back of BALB/c mice skin. Recombinant reconstituted fibrinlysin does not produce hemorrhagic activity in mice. Only when high doses (>100 μg) were used was a small hemorrhagic spot observed (less than 0.3 cm) (FIG. 15).

1. An isolated nucleic acid molecule consisting of the nucleotide sequence of SEQ ID NO:1, SEQ ID NO:3, or SEQ ID NO:5.

2. An isolated nucleic acid molecule comprising the nucleotide sequence of SEQ ID NO:1, or a degenerate variant of SEQ ID NO:1.

3. An isolated nucleic acid molecule comprising the nucleotide sequence of SEQ ID NO:3, or a degenerate variant of SEQ ID NO:3.

4. An isolated nucleic acid molecule comprising the nucleotide sequence of SEQ ID NO:5, or a degenerate variant of SEQ ID NO:5.

5. An isolated nucleic acid molecule comprising a sequence that encodes a polypeptide with the amino acid sequence of SEQ ID NO:2, SEQ ID NO:4, or SEQ ID NO:6.

6. An isolated nucleic acid molecule comprising a sequence that hybridizes under stringent conditions to a hybridization probe the nucleotide sequence of which consists of SEQ ID NO:1, or the complement of SEQ ID NO:1.

7. An isolated nucleic acid molecule comprising a sequence that hybridizes under stringent conditions to a hybridization probe the nucleotide sequence of which consists of SEQ ID NO:3, or the complement of SEQ ID NO:3.

8. An isolated nucleic acid molecule comprising a sequence that hybridizes under stringent conditions to a hybridization probe the nucleotide sequence of which consists of SEQ ID NO:5, or the complement of SEQ ID NO:5.

9. An isolated nucleic acid molecule comprising a sequence at least 80%, 90%, 95%, 97%, 98%, or 99% identical to SEQ ID NO:1.

10. An isolated nucleic acid molecule comprising a sequence at least 80%, 90%, 95%, 97%, 98%, or 99% identical to SEQ ID NO:3.

11. An isolated nucleic acid molecule comprising a sequence at least 80%, 90%, 95%, 97%, 98%, or 99% identical to SEQ ID NO:5.

12. An isolated nucleic acid molecule comprising a sequence that encodes a polypeptide the amino acid sequence of which is at least 99% identical to SEQ ID:2 or SEQ ID NO:4.

13. An isolated nucleic acid molecule comprising a sequence that encodes a polypeptide having the sequence of SEQ ID NO:2, or SEQ ID NO:2 with conservative amino acid substitutions.

14. An isolated nucleic acid molecule comprising a sequence that encodes a polypeptide having the sequence of SEQ ID NO:4, or SEQ ID NO:4 with conservative amino acid substitutions.

15. An isolated nucleic acid molecule comprising a sequence that encodes a polypeptide having the sequence of SEQ ID NO:6, or SEQ ID NO:6 with conservative amino acid substitutions.

16. An expression vector comprising the nucleic acid molecule of claim 13 operably linked to an expression control sequence.

17. An expression vector comprising the nucleic acid molecule of claim 14 operably linked to an expression control sequence.

18. An expression vector comprising the nucleic acid molecule of claim 15 operably linked to an expression control sequence.

19. A host cell comprising the vector of claim 16, 17, or 18.

20. A host cell transfected with the vector of claim 16, or a progeny of said cell.

21. A host cell transfected with the vector of claim 17, or a progeny of said cell.

22. A host cell transfected with the vector of claim 18, or a progeny of said cell.

23. A method of producing a protein, comprising culturing the cell of claims 20, 21, or 22 under conditions permitting expression of the polypeptide.

24. A method of producing a protein, comprising culturing the host cell of claims 20, 21, or 22 under conditions permitting expression under the control of the expression control sequence, and purifying the polypeptide from the cell or the medium of the cell.

25. A purified polypeptide, the amino acid sequence of which consists of SEQ ID NO:2, SEQ ID NO:4, or SEQ ID NO: 6.

26. A purified polypeptide, the amino acid sequence of which comprises SEQ ID NO:2, or SEQ ID NO:2 with at least one conservative amino acid substitution.

27. A purified polypeptide, the amino acid sequence of which comprises SEQ ID NO:4, or SEQ ID NO:4 with at least one conservative amino acid substitution.

28. A purified polypeptide, the amino acid sequence of which comprises SEQ ID NO:6, or SEQ ID NO:6 with at least one conservative amino acid substitution.

29. A purified polypeptide, the amino acid sequence of which comprises SEQ ID NO:2, or biologically active portions thereof.

30. A purified polypeptide that binds specifically to an antibody that binds specifically to a protein encoded by SEQ ID NO: 2 or SEQ ID NO:8.

31. An antibody that binds specifically to a protein encoded by SEQ ID NO:2, SEQ ID NO:4 or SEQ ID NO:8.

32. A method of making an antibody, comprising immunizing a non-human animal with an immunogenic fragment of a protein encoded by SEQ ID NO:2, SEQ ID NO:4 or SEQ ID NO:8.

33. A method of making an antibody, comprising providing a hybridoma cell that produces a monoclonal antibody specific for protein encoded by SEQ ID NO:2 or SEQ ID NO:8, and culturing the cell under conditions that permit production of the monoclonal antibody.

34. A method of purifying protein encoded by SEQ ID NO:2 or SEQ ID NO:4 from a biological sample containing protein Mucroslysin, comprising:
a. providing an affinity matrix comprising the antibody that binds specifically to SEQ ID NO:2 or SEQ ID NO: 4 bound to a solid support;
b. contacting the biological sample with the affinity matrix, to produce an affinity matrix-protein Mucroslysin complex;
c. separating the affinity matrix-protein Mucroslysin complex from the remainder of the biological sample; and
d. releasing protein Mucroslysin from the affinity matrix.

35. A method of identifying a compound that inhibits the lysis of fibrinogen by Mucroslysin, the method comprising:
a. incubating the polypeptide of claim 25 or 37 with fibrinogen and a test compound; and
b. determining whether the lysis of fibrinogen in the presence of the test compound is decreased.

36. A method of identifying a compound that increases the lysis of fibrinogen by Mucroslysin, the method comprising:
a. incubating the polypeptide of claim 25 or 37 with fibrinogen and a test compound; and
b. determining whether the lysis of fibrinogen in the presence of the test compound is increased.

37. A purified polypeptide, the amino acid sequence of which comprises a sequence at least 99% identical to SEQ ID NO:8.

38. A method of inducing fibrinogenolytic activity, the method comprising:
a. providing the polypeptide of claim 25 or 37; and
b. delivering said polypeptide to an in vitro or in vivo environment containing fibrinogen.

39. An article of manufacture, comprising packaging material and a agent contained within said packaging material, wherein said agent is effective for lysing fibrinogen, and wherein said packaging material comprises a label which indicates that said agent can be used for lysing fibrinogen, and wherein said pharmaceutical agent comprises the polypeptide of claim 25 or 37.

40. An article of manufacture comprising, packaged together:
a. a vessel containing the polypeptide of claim 25 or 37; and
b. instructions for use of polypeptide of claim 25 or 37 for the treatment of occlusive thrombi in a method comprising (a) identifying a patient suspected of having occlusive thrombi, and (b) administering an effective amount of said polypeptide to the patient.

41. An article of manufacture comprising packaging material and, contained within the packaging material, polypeptide of claim 25 or 37, wherein the packaging material comprises a label that indicates that said polypeptide can be used for treating occlusive thrombi in a patient.

42. An isolated nucleic acid molecule the sequence of which comprises SEQ ID NO:5, operably linked to a nucleic acid sequence of metalloproteinase domain of snake venom protein family.

43. A purified polypeptide the sequence of which comprises SEQ ID NO:6, connected to a polypeptide of metalloproteinase domain of snake venom protein family.

44. A fusion protein comprising the purified polypeptide of claim 26.

45. A fusion protein comprising the purified polypeptide of claim 27.

46. A fusion protein comprising the purified polypeptide of claim 37.


SEQUENCE LISTING (8 sequences)

SEQ ID NO: 1 - DNA, 1389 bp, Trimeresurus mucrosquamatus
  exon (1)..(1389)
agctctataatcctggaatctgggaacgttaatgattatgaagtagtgtatccacgaaaagtcagtgcattgcccaaaggagcagttcagccaaagtatgaagacgccatgcaatatgaatttaaagtgaatggagaggcagtggtccttcacctggaaaaaaataaaggacttttttcagaagattacagcgagactcattattcccctgatggcagagaaattacaacatacccctcggttgaggatcactgctattatcatggacgcatccacaatgacgctgactcaactgcaagcatcagtgcatgcgatggtttgaaaggatatttcaagcttcaaggggagacgtaccctattgaacccttggagctttccgacagtgaagcccatgcagtcttcaaatacgaaaatgtagaaaaagaggatgaggcccccaaaatgtgtggggtaacccagaattgggaatcagatgagtccatcaaaaaggcctctcagttatatcttactcctgaacaacaaagattcccccaaagatacattaagcttgcaatagttgtggaccatggaatgtacaccaaatacagtagcaattttaaaaagataagaaaaagggtacatcaaatggtcagcaatataaatgagatgtgcagacctctgaatattgctataacactggctctcctagacgtttggtccgaaaaagatttcattaccgtgcaggcagacgcgcctactactgcgggcttatttggagactggagagagagagtcttgctgaagaagaaaaatcatgatcatgctcagttactcacggacactaacttcgctagaaacactataggatgggcttacgtgggccgcatgtgcgatgaaaagtattctgtagcagttgttaaggatcatagctcaaaggtttttatggttgcagttacaatgacccatgagctcggtcataatctgggcatggaacacgatgataaagataagtgtaaatgtgacacatgcattatgtctgccgtgataagcgataaacaatccaaactgttcagcgattgtagtaaggattattaccagacgtttcttactaatgataacccacaatgcattctcaatgcacccttgagaacagatactgtttcaactccagtttctggaaatgaatttttggaggcgggagaagaatgtgactgtggctctcctgaaaatccgtgctgcgatgctgcaacctgtaaactgagaccaggggcgcagtgtgcagaaggactgtgttgtgaccagtgcagatttaagaaaaaaagaacaatatgccggagagcaaggggtgataacccggatgaccgctgcactggccaatctgctgactgtcccagaaatggcctctatggc

SEQ ID NO: 2 - protein, 463 aa, Trimeresurus mucrosquamatus
  PEPTIDE (1)..(463)
SSIILESGNVNDYEVVYPRKVSALPKGAVQPKYEDAMQYEFKVNGEAVVLHLEKNKGLFSEDYSETHYSPDGREITTYPSVEDHCYYHGRIHNDADSTASISACDGLKGYFKLQGETYPIEPLELSDSEAHAVFKYENVEKEDEAPKMCGVTQNWESDESIKKASQLYLTPEQQRFPQRYIKLAIVVDHGMYTKYSSNFKKIRKRVHQMVSNINEMCRPLNIAITLALLDVWSEKDFITVQADAPTTAGLFGDWRERVLLKKKNHDHAQLLTDTNFARNTIGWAYVGRMCDEKYSVAVVKDHSSKVFMVAVTMTHELGHNLGMEHDDKDKCKCDTCIMSAVISDKQSKLFSDCSKDYYQTFLTNDNPQCILNAPLRTDTVSTPVSGNEFLEAGEECDCGSPENPCCDAATCKLRPGAQCAEGLCCDQCRFKKKRTICRRARGDNPDDRCTGQSADCPRNGLYG

SEQ ID NO: 3 - DNA, 609 bp, Trimeresurus mucrosquamatus
  exon (1)..(609)
gaacaacaaagattcccccaaagatacattaagcttgcaatagttgtggaccatggaatgtacaccaaatacagtagcaattttaaaaagataagaaaaagggtacatcaaatggtcagcaatataaatgagatgtgcagacctctgaatattgctataacactggctctcctagacgtttggtccgaaaaagatttcattaccgtgcaggcagacgcgcctactactgcgggcttatttggagactggagagagagagtcttgctgaagaagaaaaatcatgatcatgctcagttactcacggacactaacttcgctagaaacactataggatgggcttacgtgggccgcatgtgcgatgaaaagtattctgtagcagttgttaaggatcatagctcaaaggtttttatggttgcagttacaatgacccatgagctcggtcataatctgggcatggaacacgatgataaagataagtgtaaatgtgacacatgcattatgtctgccgtgataagcgataaacaatccaaactgttcagcgattgtagtaaggattattaccagacgtttcttactaatgataacccacaatgcattctcaatgcaccc

SEQ ID NO: 4 - protein, 203 aa, Trimeresurus mucrosquamatus
  PEPTIDE (1)..(203)
EQQRFPQRYIKLAIVVDHGMYTKYSSNFKKIRKRVHQMVSNINEMCRPLNIAITLALLDVWSEKDFITVQADAPTTAGLFGDWRERVLLKKKNHDHAQLLTDTNFARNTIGWAYVGRMCDEKYSVAVVKDHSSKVFMVAVTMTHELGHNLGMEHDDKDKCKCDTCIMSAVISDKQSKLFSDCSKDYYQTFLTNDNPQCILNAP

SEQ ID NO: 5 - DNA, 213 bp, Trimeresurus mucrosquamatus
  exon (1)..(213)
ggagaagaatgtgactgtggctctcctgaaaatccgtgctgcgatgctgcaacctgtaaactgagaccaggggcgcagtgtgcagaaggactgtgttgtgaccagtgcagatttaagaaaaaaagaacaatatgccggagagcaaggggtgataacccggatgaccgctgcactggccaatctgctgactgtcccagaaatggcctctatggc

SEQ ID NO: 6 - protein, 71 aa, Trimeresurus mucrosquamatus
  PEPTIDE (1)..(71)
GEECDCGSPENPCCDAATCKLRPGAQCAEGLCCDQCRFKKKRTICRRARGDNPDDRCTGQSADCPRNGLYG

SEQ ID NO: 7 - DNA, 2139 bp, Trimeresurus mucrosquamatus
  exon (96)..(1541)
ggttccttgcttttcatagtcaacagaggaagagctcaggttggcttgaaagcaggaagagattgcctgtcttccagccaaatccagcctccaaaatgattgaagttctcttggtaaccatatgcttagcagtctttccttatcaagggagctctataatcctggaatctgggaacgttaatgattatgaagtagtgtatccacgaaaagtcagtgcattgcccaaaggagcagttcagccaaagtatgaagacgccatgcaatatgaatttaaagtgaatggagaggcagtggtccttcacctggaaaaaaataaaggacttttttcagaagattacagcgagactcattattcccctgatggcagagaaattacaacatacccctcggttgaggatcactgctattatcatggacgcatccacaatgacgctgactcaactgcaagcatcagtgcatgcgatggtttgaaaggatatttcaagcttcaaggggagacgtaccctattgaacccttggagctttccgacagtgaagcccatgcagtcttcaaatacgaaaatgtagaaaaagaggatgaggcccccaaaatgtgtggggtaacccagaattgggaatcagatgagtccatcaaaaaggcctctcagttatatcttactcctgaacaacaaagattcccccaaagatacattaagcttgcaatagttgtggaccatggaatgtacaccaaatacagtagcaattttaaaaagataagaaaaagggtacatcaaatggtcagcaatataaatgagatgtgcagacctctgaatattgctataacactggctctcctagacgtttggtccgaaaaagatttcattaccgtgcaggcagacgcgcctactactgcgggcttatttggagactggagagagagagtcttgctgaagaagaaaaatcatgatcatgctcagttactcacggacactaacttcgctagaaacactataggatgggcttacgtgggccgcatgtgcgatgaaaagtattctgtagcagttgttaaggatcatagctcaaaggtttttatggttgcagttacaatgacccatgagctcggtcataatctgggcatggaacacgatgataaagataagtgtaaatgtgacacatgcattatgtctgccgtgataagcgataaacaatccaaactgttcagcgattgtagtaaggattattaccagacgtttcttactaatgataacccacaatgcattctcaatgcacccttgagaacagatactgtttcaactccagtttctggaaatgaatttttggaggcgggagaagaatgtgactgtggctctcctgaaaatccgtgctgcgatgctgcaacctgtaaactgagaccaggggcgcagtgtgcagaaggactgtgttgtgaccagtgcagatttaagaaaaaaagaacaatatgccggagagcaaggggtgataacccggatgaccgctgcactggccaatctgctgactgtcccagaaatggcctctatggctaaccaacaatggagctggaccacaatgcattctcaatgcagacccttgagaacagatactgtttcaactccagtttctggaaatgaacttttgtaggcgggagaagattgtgactgtggctctccttcaatctgtagcaacaggcagtgttgatgtgactctacagcatactaattaaccactggcttctctcagatttgattttggagatcctccttccagaaggtttgccttccctctagtccaaagagatccatctgcctgcatcctactagtaaatcacccttagctttcatatggaatctaaattatgcaatatttcttttccatatttaatctgtttacctcttgctgtaatcaaacctttttcccaccacaaagctccatgggcatgtacaacaccaagagctgttttgctgtcaagaaaaaaaaatggccattttacattttacatttgccaattgcaaagtacatttaatgcaacaagttctgcctttagagctggtgtattcgaagtgaatgcttcctctcccaaaatttcatgctggctttccaagatgtaactgcttccatcaataaactcactattctcattcaaaa

SEQ ID NO: 8 - protein, 481 aa, Trimeresurus mucrosquamatus
  PEPTIDE (1)..(481)
MIEVLLVTICLAVFPYQGSSIILESGNVNDYEVVYPRKVSALPKGAVQPKYEDAMQYEFKVNGEAVVLHLEKNKGLFSEDYSETHYSPDGREITTYPSVEDHCYYHGRIHNDADSTASISACDGLKGYFKLQGETYPIEPLELSDSEAHAVFKYENVEKEDEAPKMCGVTQNWESDESIKKASQLYLTPEQQRFPQRYIKLAIVVDHGMYTKYSSNFKKIRKRVHQMVSNINEMCRPLNIAITLALLDVWSEKDFITVQADAPTTAGLFGDWRERVLLKKKNHDHAQLLTDTNFARNTIGWAYVGRMCDEKYSVAVVKDHSSKVFMVAVTMTHELGHNLGMEHDDKDKCKCDTCIMSAVISDKQSKLFSDCSKDYYQTFLTNDNPQCILNAPLRTDTVSTPVSGNEFLEAGEECDCGSPENPCCDAATCKLRPGAQCAEGLCCDQCRFKKKRTICRRARGDNPDDRCTGQSADCPRNGLYG